(19) United States
(12) Patent Application Publication
(10) Pub. No.: US 2019/0391581 A1
(43) Pub. Date: Dec. 26, 2019

(54) Passenger Health Monitoring and Intervention for Autonomous Vehicles

(71) Applicant: Uber Technologies, Inc., San Francisco, CA (US)

(72) Inventors: Anthony Alfred Vardaro, Pittsburgh, PA (US); Richard Arthur Rattanni, Verona, PA (US)

(21) Appl. No.: 16/040,806

(22) Filed: Jul. 20, 2018

Related U.S. Application Data
(60) Provisional application No. 62/690,032, filed on Jun. 26, 2018.

(51) Int. Cl. G05D 1/00; G16H 50/20; A61B 5/18; A61B 5/00; A61B 5/0205

(52) U.S. Cl. CPC G05D 1/0088; G05D 1/0055; G16H 50/20; A61B 5/18; A61B 5/01; A61B 5/02055; A61B 5/6893; A61B 5/7282; A61B 5/0059; A61B 5/486

(57) ABSTRACT

Systems, methods, tangible non-transitory computer-readable media, and devices for operating an autonomous vehicle are provided. For example, a vehicle computing system can receive sensor data and vehicle data from sensors of a vehicle. The sensor data can be associated with states of passengers in a passenger compartment of the vehicle. The vehicle data can be associated with states of the vehicle. The vehicle computing system can determine, based on the sensor data and the vehicle data, when the states of the passengers are associated with health conditions. Actions for the vehicle to perform can be determined based on the health conditions associated with the passengers. Furthermore, the vehicle computing system can generate control signals to control performance of the actions by the vehicle.

### RELATED APPLICATION

The present application is based on and claims benefit of U.S. Provisional Patent Application No. 62/690,032 having a filing date of Jun. 26, 2018, which is incorporated by reference herein.

### FIELD

The present disclosure relates generally to monitoring states of a passenger of an autonomous vehicle including states associated with health conditions.

### BACKGROUND

The operation of vehicles, including autonomous vehicles, can involve a variety of changes in the state of passengers carried by the vehicle. For example, the state of passengers can change based on differences between individual passengers. Further, the vehicle can carry passengers that respond to the changes in the way the vehicle is operated. As the vehicle operates in various different environments under different conditions, the states of passengers can change in different ways. Accordingly, there exists a need for a way to more effectively determine various states including the states of passengers inside the vehicle, thereby improving the experience of traveling inside the vehicle.

### SUMMARY

Aspects and advantages of embodiments of the present disclosure will be set forth in part in the following description, or may be learned from the description, or may be learned through practice of the embodiments.

An example aspect of the present disclosure is directed to a computer-implemented method of autonomous vehicle operation. The computer-implemented method can include receiving, by a computing system including one or more computing devices, sensor data and vehicle data, from one or more sensors of a vehicle. The sensor data can be associated with one or more states of one or more passengers in a passenger compartment of the vehicle. Further, the vehicle data can be associated with one or more states of the vehicle. The method can include determining, by the computing system, based at least in part on the sensor data and the vehicle data, when the one or more states of the one or more passengers are associated with one or more health conditions. The method can also include determining, by the computing system, based at least in part on the one or more health conditions associated with the one or more passengers, one or more actions to be performed by the vehicle. Furthermore, the method can include generating, by the computing system, one or more control signals to control performance of the one or more actions by the vehicle.

Another example aspect of the present disclosure is directed to one or more tangible non-transitory computer-readable media storing computer-readable instructions that when executed by one or more processors cause the one or more processors to perform operations. The operations can include receiving sensor data and vehicle data, from one or more sensors of a vehicle. The sensor data can be associated with one or more states of one or more passengers in a passenger compartment of the vehicle. Further, the vehicle data can be associated with one or more states of the vehicle. The operations can include determining, based at least in part on the sensor data and the vehicle data, when the one or more states of the one or more passengers are associated with one or more health conditions. The operations can also include determining, based at least in part on the one or more health conditions associated with the one or more passengers, one or more actions to be performed by the vehicle. Furthermore, the operations can include generating one or more control signals to control performance of the one or more actions by the vehicle.

Another example aspect of the present disclosure is directed to a vehicle including one or more processors and a memory including one or more computer-readable media. The memory can store computer-readable instructions that when executed by the one or more processors can cause the one or more processors to perform operations. The operations can include receiving sensor data and vehicle data, from one or more sensors of a vehicle. The sensor data can be associated with one or more states of one or more passengers in a passenger compartment of the vehicle. Further, the vehicle data can be associated with one or more states of the vehicle. The operations can include determining, based at least in part on the sensor data and the vehicle data, when the one or more states of the one or more passengers are associated with one or more health conditions. The operations can also include determining, based at least in part on the one or more health conditions associated with the one or more passengers, one or more actions to be performed by the vehicle. Furthermore, the operations can include generating one or more control signals to control performance of the one or more actions by the vehicle.

Other example aspects of the present disclosure are directed to other systems, methods, vehicles, apparatuses, tangible non-transitory computer-readable media, and devices for monitoring the state of an autonomous vehicle and a passenger of an autonomous vehicle. These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 depicts an example system according to example embodiments of the present disclosure;

FIG. 2 depicts a top view of the interior of a vehicle according to example embodiments of the present disclosure;

FIG. 3 depicts an example of passenger seating area sensors according to example embodiments of the present disclosure;

FIG. 4 depicts an example of a computing system including a display device according to example embodiments of the present disclosure;

FIG. 5 depicts a flow diagram of an example method of passenger monitoring and intervention according to example embodiments of the present disclosure;

FIG. 6 depicts a flow diagram of an example method of passenger monitoring and intervention according to example embodiments of the present disclosure;

FIG. 7 depicts a flow diagram of an example method of passenger monitoring and intervention according to example embodiments of the present disclosure;

FIG. 8 depicts a flow diagram of an example method of passenger monitoring and intervention according to example embodiments of the present disclosure;

FIG. 9 depicts a flow diagram of an example method of passenger monitoring and intervention according to example embodiments of the present disclosure;

FIG. 10 depicts a flow diagram of an example method of passenger monitoring and intervention according to example embodiments of the present disclosure;

FIG. 11 depicts a flow diagram of an example method of passenger monitoring and intervention according to example embodiments of the present disclosure;

FIG. 12 depicts a flow diagram of an example method of passenger monitoring and intervention according to example embodiments of the present disclosure;

FIG. 13 depicts a flow diagram of an example method of passenger monitoring and intervention according to example embodiments of the present disclosure; and

FIG. 14 depicts an example system according to example embodiments of the present disclosure.

### DETAILED DESCRIPTION

Example aspects of the present disclosure are directed to a vehicle (e.g., an autonomous vehicle, a semi-autonomous vehicle, or a manually operated vehicle) performing operations based on a state (e.g., a physiological condition) of a passenger in the vehicle. The disclosed technology can perform one or more actions to expedite and/or facilitate medical attention being provided to a passenger experiencing a health condition that is in need of timely assistance.

In particular, aspects of the present disclosure include a computing system (e.g., a vehicle computing system including one or more computing devices that can be configured to monitor a vehicle passenger compartment and control one or more vehicle systems) that can receive sensor data associated with the state of a vehicle's passenger compartment, determine passenger states (e.g., passenger heart rate, respiratory rate, and/or movement profile) of passengers in the passenger compartment that are associated with health conditions (e.g., cardiac arrest, stroke, or other health conditions that reduce or eliminate a passenger's ability to self-report their health condition including self-reporting through words, gestures, or interactions with an interface of the vehicle), determine actions for the vehicle to perform based on the health conditions, and generate control signals so that the vehicle can perform the actions (e.g., send a request for emergency medical services when the passenger state is associated with a health condition corresponding to cardiac arrest).

By way of example, a vehicle computing system can receive sensor data from one or more sensors (e.g., one or more biometric sensors worn by the one or more passengers and one or more vehicle system sensors that detect motion characteristics of the vehicle) as a vehicle travels on a road. For example, the vehicle computing system can use one or more physiological state determination techniques (e.g., rules based techniques and/or a machine-learned model) to determine when a health condition is being experienced by one or more passengers of the vehicle. For example, the occurrence of the health condition can be based on one or more physiological states (e.g., heart rate, blood pressure, and/or movement patterns) of the one or more passengers. Further, the computing system can determine an action to perform based on the health condition of the passenger. For example, the vehicle can drive the passenger to the nearest hospital when the computing system determines that the passenger is experiencing a health condition. As such, the disclosed technology can perform one or more actions to expedite and/or facilitate medical attention being provided to a passenger experiencing a health condition requires timely assistance.

The disclosed technology can include a vehicle computing system (e.g., one or more computing devices that includes one or more processors and a memory) that can process, generate, and/or exchange (e.g., send and/or receive) signals or data, including signals or data exchanged with various devices including one or more vehicles, vehicle components (e.g., engine, brakes, steering, and/or transmission), and/or remote computing devices (e.g., one or more smart phones and/or wearable devices). For example, the vehicle computing system can exchange one or more signals (e.g., electronic signals) or data with one or more vehicle systems including biometric monitoring systems (e.g., one or more heart rate sensors, respiratory sensors, thermal sensors, blood pressure sensors, grip strength sensors, motion sensors, galvanic skin response sensors, and/or pupillary dilation sensors); passenger compartment systems (e.g., cabin temperature monitoring systems, cabin humidity monitoring systems, cabin ventilation systems, and/or seat sensors including headrest sensors); vehicle access systems (e.g., door, window, and/or trunk systems); illumination systems (e.g., headlights, internal lights, signal lights, and/or tail lights); sensor systems (e.g., sensors that generate output based on the state of the physical environment inside the vehicle and/or external to the vehicle, including one or more light detection and ranging (LIDAR) devices, cameras, microphones, radar devices, and/or sonar devices); communication systems (e.g., wired or wireless communication systems that can exchange signals or data with other devices); navigation systems (e.g., devices that can receive signals from GPS, GLONASS, or other systems used to determine a vehicle's geographical location); notification systems (e.g., devices used to provide notifications to passengers, including one or more display devices, status indicator lights, and/or audio output systems); braking systems (e.g., brakes of the vehicle including mechanical and/or electric brakes); propulsion systems (e.g., motors or engines including internal combustion engines or electric engines); and/or steering systems used to change the path, course, or direction of travel of the vehicle.

In some embodiments, the vehicle computing system can include a machine-learned model (e.g., a machine-learned passenger state model that is stored in one or more memory devices of the vehicle computing system) that is trained to receive input data which can include the sensor data and/or vehicle data and which can provide (e.g., generate, make available, etc.) an output that can include one or more health condition predictions associated with one or more health conditions. Further, the vehicle computing system can receive the output provided by the machine-learned model and can use the output from the machine-learned model to perform one or more actions (e.g., contacting an emergency medical provider when a health condition is detected).

In some embodiments, the vehicle computing system can access a machine-learned model that has been generated and/or trained using training data including a plurality of classified features and a plurality of classified health condition labels. In some embodiments, the plurality of classified features can be extracted from one or more sensor outputs (e.g., biometric sensor outputs including heart rate, blood pressure, respiratory rate, and/or body temperature) received from one or more sensors that are used to detect one or more states of a person (e.g., a passenger of a vehicle).

When the machine-learned model has been trained, the machine-learned model can associate the plurality of classified features with one or more classified health condition labels that are used to classify or categorize the state of a person associated with the plurality of classified features. In some embodiments, as part of the process of training the machine-learned model, the differences in correct classification output between a machine-learned model (that outputs the one or more classified health condition labels) and a set of classified health condition labels associated with training data that has previously been correctly identified (e.g., ground truth labels), can be processed using an error loss function that can determine a set of probability distributions based on repeated classification of the same set of training data. As such, the effectiveness (e.g., the rate of correct identification of health conditions) of the machine-learned model can be improved over time.

The vehicle computing system can access the machine-learned model in a variety of ways including exchanging (sending and/or receiving via a network) data or information associated with a machine-learned model that is stored on a remote computing device; and/or accessing a machine-learned model that is stored locally (e.g., in one or more storage devices of the vehicle computing system and/or the vehicle).

The plurality of classified features can be associated with one or more values that can be analyzed individually and/or in various aggregations. Analysis of the one or more values associated with the plurality of classified features can include determining a mean, mode, median, variance, standard deviation, maximum, minimum, and/or frequency of the one or more values associated with the plurality of classified features. Further, processing and/or analysis of the one or more values associated with the plurality of classified features can include comparisons of the differences or similarities between the one or more values. For example, one or more bodily movements (e.g., jerky movements) associated with a health condition experienced by a passenger can be associated with a range of bodily movements that are different from the range of bodily movements associated with a passenger that is in a state of good health.

In some embodiments, the plurality of classified features can include a range of sounds associated with the plurality of training subjects (e.g., people who have volunteered to provide test data based on their physical responses), a range of temperatures associated with the plurality of training subjects, a range of body positions associated with the plurality of training subjects, a range of gestures and/or movement patterns associated with the plurality of training subjects, a range of heart rates associated with the plurality of training subjects, a range of respiratory rates associated with the plurality of training subjects, a range of blood pressures associated with the plurality of training subjects, physical characteristics (e.g., age, gender, height, weight, and/or mass) of the plurality of training subjects. The plurality of classified features can be based at least in part on the output from one or more sensors that have captured sensor data from the plurality of training subjects at various times of day and/or in different vehicle conditions (e.g., a warm passenger compartment, a cold passenger compartment, a passenger compartment moving at high velocity, and/or a crowded passenger compartment) and/or environmental conditions (e.g., bright sunlight, rain, overcast conditions, darkness, and/or thunder storms).

The machine-learned model can be generated based at least in part on one or more classification processes or classification techniques. The one or more classification processes or classification techniques can include one or more computing processes performed by one or more computing devices based at least in part on sensor data associated with physical outputs from a sensor device. The one or more computing processes can include the classification (e.g., allocation or sorting into different groups or categories) of the physical outputs from the sensor device, based at least in part on one or more classification criteria associated with one or more health conditions. In some embodiments, the machine-learned model can include a convolutional neural network, a recurrent neural network, a recursive neural network, gradient boosting, a support vector machine, and/or a logistic regression classifier.

The vehicle computing system can receive sensor data and vehicle data, from one or more sensors of a vehicle. The sensor data can be associated with one or more states of one or more passengers in a passenger compartment of the vehicle. Further, the vehicle data can be associated with one or more states of the vehicle. For example, the vehicle computing system can include one or more transceivers that can send and/or receive one or more signals (e.g., signals transmitted wirelessly and/or via a wired connection) that include the sensor data and/or the vehicle data.

In some embodiments, the vehicle data can include information associated with a velocity of the vehicle, an acceleration of the vehicle, a deceleration of the autonomous vehicle, a centrifugal force on the passenger compartment of the vehicle, a temperature of the passenger compartment of the vehicle, and/or a humidity of the passenger compartment of the vehicle.

In some embodiments, the one or more sensors can include one or more heart rate sensors, one or more respiratory rate sensors, one or more blood pressure sensors, one or more image sensors, one or more microphones, and/or one or more thermal sensors.

The vehicle computing system can determine, based at least in part on the sensor data and the vehicle data, when the one or more states of the one or more passengers are associated with one or more health conditions. For example, the vehicle computing system can determine that the state of a passenger is associated with a health condition (e.g., when the passenger exhibits arm and leg movements that correspond to a seizure, such as multiple jerky movements).

In some embodiments, the one or more health conditions can include a dyspneic state (e.g., shortness of breath), a state of emesis (e.g., vomiting), a seizure state (e.g., an epileptic seizure), a cardiac arrest state, or a stroke state (e.g., an ischemic stroke and/or hemorrhagic stroke).

In some embodiments, determining when the sensor data satisfies one or more health response criteria associated with an occurrence of the one or more health conditions can be utilized in determining when the one or more states of the one or more passengers are associated with one or more health conditions. For example, the sensor data satisfying the one or more health response criteria can include a passenger heart rate exceeding a heart rate threshold or a passenger blood pressure being lower than a blood pressure threshold.

In some embodiments, determining when the one or more states of the one or more passengers are associated with one or more health conditions can include adjusting the one or more health response criteria based at least in part on the vehicle data. For example, a heart rate threshold can be adjusted upwards to account for the acceleration of the vehicle carrying the passenger.

In some embodiments, the number of the one or more passengers in the vehicle can be determined. For example, sensor data from one or more cameras in the vehicle can be used by the vehicle computing system to determine the number of passengers in the vehicle. Further, the vehicle computing system can adjust the one or more health response criteria based at least in part on the number of the one or more passengers in the vehicle. For example, the vehicle computing system can adjust thermal thresholds based on the increased body heat resulting from multiple passengers in the same vehicle.

In some embodiments, determining when the one or more states of the one or more passengers are associated with one or more health conditions can include determining a respiratory pattern for each of the one or more passengers based at least in part on one or more changes in air pressure inside the passenger compartment of the vehicle and/or one or more sensor outputs of a motion sensing respiration sensor in a safety restraint device of the vehicle. Further, the vehicle computing system can compare the respiratory pattern for each of the one or more passengers to a set of respiratory rates associated with the one or more health conditions.

For example, the vehicle computing system can determine a passenger's rate of respiration based at least in part on the movement of a pressure sensor in a seat belt that is placed across the passenger's chest (e.g., a three-point seat belt with a strap across the waist and another strap crossing the passenger's chest from waist to shoulder). Further, the one or more health conditions can be associated with various rates of respiration (e.g., shortness of breath can correspond to a respiratory pattern that includes a rapid respiratory rate).

In some embodiments, determining, based at least in part on the sensor data, one or more characteristics of one or more motions of a passenger of the one or more passengers can be utilized in determining when the one or more states of the one or more passengers are associated with one or more health conditions. Further, the vehicle computing system can determine when the one or more motions of the passenger satisfy one or more passenger motion criteria associated with the one or more health conditions. For example, erratic movements of a passenger can be associated with health conditions including a seizure.

In some embodiments, determining when the one or more states of the one or more passengers are associated with one or more health conditions can include generating a passenger state query that can include a request for passenger state feedback based at least in part on the one or more states of the one or more passengers. The passenger state query can include one or more visual indications and/or one or more auditory indications. Further, the vehicle computing system can determine the one or more states of the one or more passengers based at least in part on the passenger state feedback. For example, the vehicle computing system can generate a passenger state query asking a passenger “Are you OK?” to which a passenger reply of “NO” can result in the performance of one or more actions to assist the passenger.

In some embodiments, the passenger state query can include requesting at least one of the one or more passengers to provide an audible response to the passenger state query, requesting at least one of the one or more passengers to gesture in response to the passenger state query, and/or requesting at least one of the one or more passengers to provide a physical input to an interface of the vehicle.

In some embodiments, determining the one or more states of the one or more passengers based at least in part on the passenger state feedback can include the vehicle computing system determining that the one or more states of the one or more passengers are associated with at least one of the one or more health conditions when a predetermined time interval has elapsed without receiving the passenger state feedback. For example, if a passenger does not respond within twenty seconds, the vehicle computing system can determine that the one or more passenger states of the one or more passengers are associated with one or more health conditions.

In some embodiments, determining when the one or more states of the one or more passengers are associated with one or more health conditions can include comparing the one or more states of the one or more passengers to aggregate passenger state data that can include one or more physiological states of one or more test passengers and one or more corresponding physical conditions of the one or more test passengers. For example, the aggregate passenger state data can include one or more states of the same passenger taking the ride or a collection of anonymized data from various passengers that have previously agreed to provide their biometric information. Further, the vehicle computing system can determine when the one or more states of the one or more passengers are within one or more threshold ranges corresponding to the one or more physiological states of the one or more test passengers.

In some embodiments, the sensor data and the vehicle data can be sent to a machine-learned passenger state model (e.g., a machine-learned passenger state model stored in a portion of a memory device of the vehicle computing system) that is trained to receive the sensor data and the vehicle data and provide (send from one portion of the vehicle computing system to another portion of the vehicle computing system) an output including one or more health conditions associated with the one or more passengers. For example, the sensor data and vehicle data can be sent to a portion of the vehicle computing system that includes a convolutional neural network that is trained to determine the occurrence of health conditions and can provide an estimate of a health condition based on inputs of sensor data and vehicle data.

In some embodiments, one or more baseline states can be determined corresponding to the one or more states of each of the one or more passengers when the one or more passengers enter the vehicle. Further, the vehicle computing system can compare the one or more states of the one or more passengers to the one or more baseline states of the one or more passengers at one or more time intervals after the one or more passengers enter the vehicle. For example, the vehicle computing system can compare the heart rates and respiration rates of passengers when the passengers enter the vehicle to their heart rates and respiration rates as the vehicle travels. In this way, the vehicle computing system can monitor the states of a passenger in transit and can determine the occurrence of a health condition when there is a sharp deviation in the passenger's states in a short period of time.

The vehicle computing system can determine, based at least in part on the one or more health conditions associated with the one or more passengers, one or more actions to be performed by the vehicle. For example, the vehicle computing system can determine that the one or more actions performed by the vehicle will include driving a passenger to a hospital when the one or more states of the passenger are determined to be associated with a stroke.

In some embodiments, a corresponding time sensitivity associated with an amount of time before requiring medical assistance can be determined. Further, the vehicle computing system can determine the one or more actions to be performed by the vehicle based at least in part on the time sensitivity associated with the least amount of time before requiring medical assistance. For example, the vehicle computing system can determine that cardiac arrest is associated with less time before requiring medical assistance than a cut finger and will perform actions responsive to cardiac arrest when both a cut finger and cardiac arrest are determined to have occurred.

The vehicle computing system can generate one or more control signals to control performance of the one or more actions by the vehicle. For example, the vehicle computing system can send control signals that activate vehicle systems including communications systems (e.g., to send messages to health care providers) and vehicle control systems to guide the vehicle to a medical facility.

In some embodiments, the one or more actions can include sending one or more signals to a medical facility, sending one or more signals to a vehicle tele-operator, and/or driving the vehicle to a destination determined by at least one of the one or more passengers. Furthermore, generating one or more control signals to control performance of the one or more actions by the vehicle can include sending one or more notifications to a predetermined recipient associated with at least one of the one or more passengers. The predetermined recipient can include a guardian and/or an emergency medical contact.

In some embodiments, generating one or more control signals to control performance of the one or more actions by the vehicle can include determining a location of the vehicle and sending one or more notifications to an emergency medical provider. The one or more notifications can include the location of the vehicle and the one or more states of the one or more passengers. For example, the vehicle computing system can send one or more notifications indicating the potential health condition of a passenger and a location of the vehicle including a latitude and longitude of the vehicle.

The systems, methods, devices, and tangible non-transitory computer readable media in the disclosed technology can provide a variety of technical effects and benefits. In particular, the disclosed technology can provide numerous benefits including improvements in the areas of health monitoring, passenger safety, and energy conservation. The disclosed technology can improve passenger safety by monitoring the physiological states of passengers and performing some actions (e.g., driving to a hospital) when the state of the passengers corresponds to a health condition (e.g., a health condition that is harmful to the passenger). For example, a passenger of a vehicle can experience a seizure when the vehicle is in transit. By determining, based on sensor data associated with the biometric state of the passenger (e.g., heart rate), the disclosed technology can determine when a passenger is experiencing a health condition and perform actions to notify a health care provider that the passenger is experiencing the health condition and facilitate putting the passenger in the care of the health care provider (e.g., providing the location of the vehicle to an ambulance or driving the passenger to a hospital).

Further, the disclosed technology can improve the overall safety of passengers by quickly determining when a passenger is experiencing a health condition and performing relevant actions to assist the passenger. For example, sensors in the vehicle can determine when a passenger's movements are erratic, which can be associated with the occurrence of a seizure. In response to determining that the passenger is experiencing a seizure the vehicle computing system can notify a health care provider. Further, the vehicle computing system can distinguish between normal states (e.g., excited laughter or discussion) and health conditions (e.g., seizures), by querying a passenger. If the passenger does not respond in a manner that indicates that the passenger is well (e.g., the passenger indicates that he or she is unwell or does not respond within a predetermined time period) the vehicle computing system can perform a remedial action. Conversely, within a range of biometric indicators, if the passenger indicates that their state is normal, the vehicle can continue to monitor the passenger without immediately performing a remedial action.

Furthermore, the disclosed technology can reduce the time needed for a passenger to receive medical care by performing one or more actions including notifying a medical facility (e.g., a hospital or clinic) of the passenger's health condition or driving to a location at which an ambulance can pick up the passenger and treat the passenger on the way to a medical facility. In this way, the burden on medical resources can be reduced by providing passengers with access to medical care before a health condition is exacerbated. Additionally, in situations when an ambulance or other emergency vehicle is not available to pick-up the passenger, the disclosed technology can drive the passenger to a medical facility so that the passenger can receive treatment posthaste.

Accordingly, the disclosed technology can provide more effective determination of the occurrence of a health condition in a passenger and performance of remedial actions through improvements in passenger monitoring, health condition determination, and performance of actions to notify and direct health care providers.

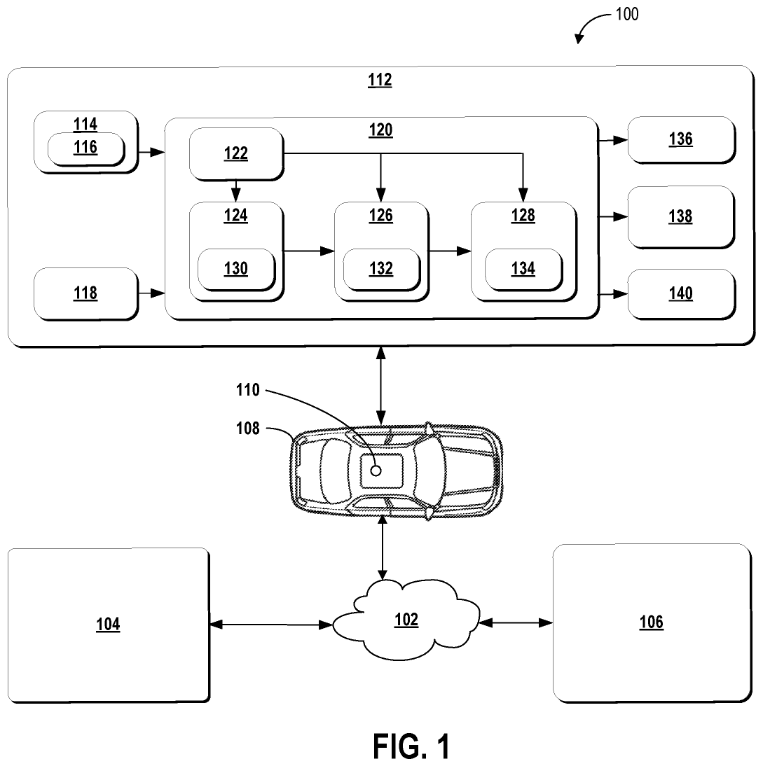

With reference now to FIGS. 1-14, example embodiments of the present disclosure will be discussed in further detail. FIG. 1 depicts a diagram of an example system 100 according to example embodiments of the present disclosure. As illustrated, FIG. 1 shows a system 100 that includes a communication network 102; an operations computing system 104; one or more remote computing devices 106; a vehicle 108; one or more passenger compartment sensors 110; a vehicle computing system 112; one or more sensors 114; sensor data 116; a positioning system 118; an autonomy computing system 120; map data 122; a perception system 124; a prediction system 126; a motion planning system 128; state data 130; prediction data 132; motion plan data 134; a communication system 136; a vehicle control system 138; and a human-machine interface 140.

The operations computing system 104 can be associated with a service provider that can provide one or more vehicle services to a plurality of users via a fleet of vehicles that includes, for example, the vehicle 108. The vehicle services can include transportation services (e.g., rideshare services), courier services, delivery services, and/or other types of services.

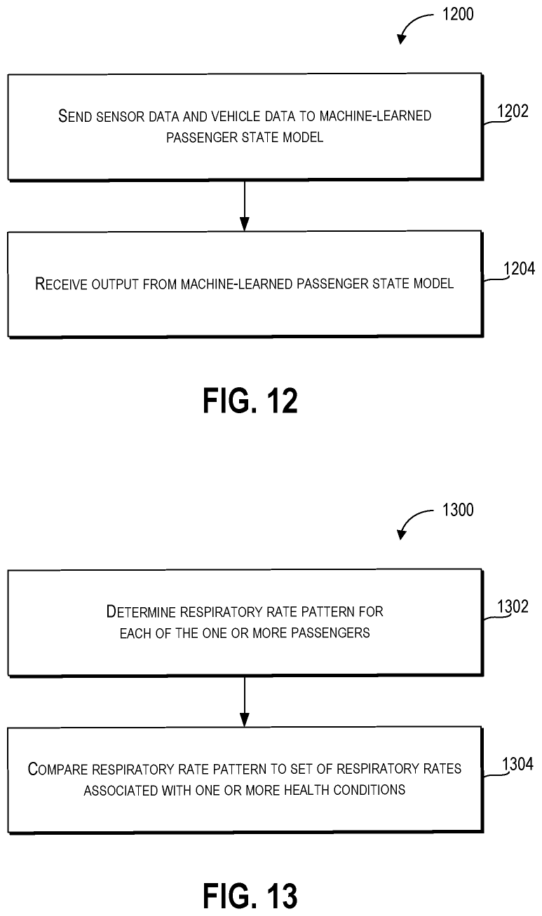

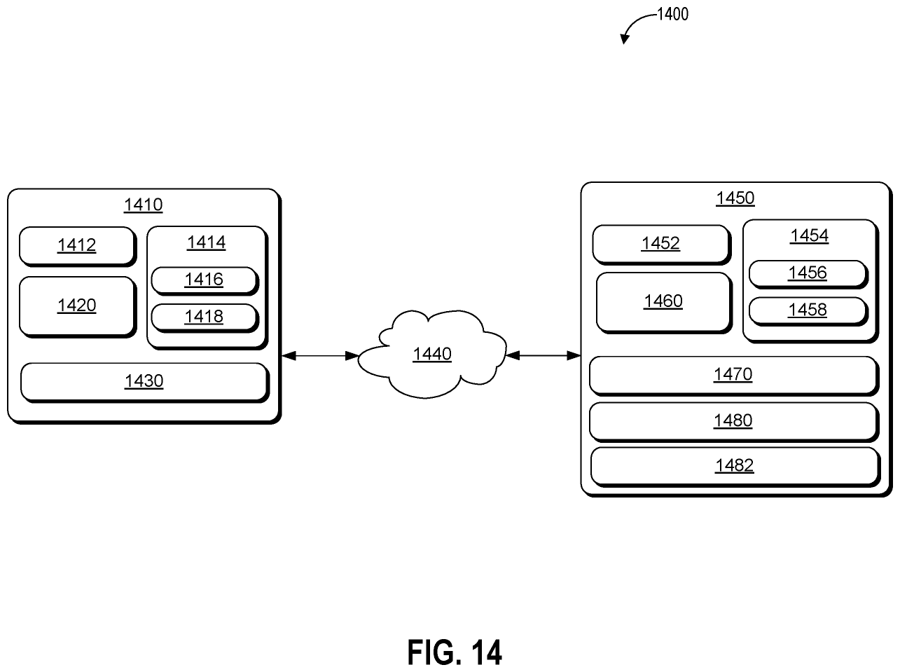

The operations computing system 104 can include multiple components for performing various operations and functions. For example, the operations computing system 104 can include and/or otherwise be associated with the one or more computing devices that are remote from the vehicle 108. The one or more computing devices of the operations computing system 104 can include one or more processors and one or more memory devices. The one or more memory devices of the operations computing system 104 can store instructions that when executed by the one or more processors cause the one or more processors to perform operations and/or functions associated with operation of a vehicle including receiving sensor data (e.g., sensor data associated with one or more states of one or more passengers in a passenger compartment of the vehicle 108) and/or vehicle data (e.g., vehicle data associated with one or more states of the vehicle 108) from sensors (e.g., the one or more sensors 114) of a vehicle (e.g., the vehicle 108); determining, based at least in part on the sensor data and/or the vehicle data, when the states of the passengers are associated with one or more health conditions; determining actions for the vehicle to perform based at least in part on the one or more health conditions associated with the passengers including health conditions (e.g., cardiac arrest and/or stroke) that interfere with or reduce a passenger's ability (e.g., the ability to speak and/or move including the ability to control limbs or hands) to self-report their health condition (e.g., communicate or describe their health condition); and generating one or more control signals to control performance of the one or more actions by the vehicle. Furthermore, in some embodiments, the operations computing system 104 can perform one or more operations including accessing one or more machine-learned models (e.g., machine-learned models that are part of the operations computing system 104 and/or a remote computing device) that include one or more features of the one or more machine-learned models 1430 depicted in FIG. 14, the one or more machine-learned models 1470 which are depicted in FIG. 14, and/or the machine-learned passenger state model described in the method 1200 depicted in FIG. 12.

For example, the operations computing system 104 can be configured to monitor and communicate with the vehicle 108 and/or its users to coordinate a vehicle service provided by the vehicle 108. To do so, the operations computing system 104 can manage a database that includes data including vehicle status data associated with the status of vehicles including the vehicle 108; and/or passenger status data associated with the status of passengers of the vehicle. The vehicle status data can include a location of a vehicle (e.g., a latitude and longitude of a vehicle), the availability of a vehicle (e.g., whether a vehicle is available to pick-up or drop-off passengers and/or cargo), or the state of objects external to a vehicle (e.g., the physical dimensions and/or appearance of objects external to the vehicle). The passenger status data can include one or more states of passengers of the vehicle including biometric or physiological states of the passengers (e.g. heart rate, blood pressure, and/or respiratory rate).

The operations computing system 104 can communicate with the one or more remote computing devices 106 and/or the vehicle 108 via one or more communications networks including the communications network 102. The communications network 102 can exchange (send or receive) signals (e.g., electronic signals) or data (e.g., data from a computing device) and include any combination of various wired (e.g., twisted pair cable) and/or wireless communication mechanisms (e.g., cellular, wireless, satellite, microwave, and radio frequency) and/or any desired network topology (or topologies). For example, the communications network 102 can include a local area network (e.g. intranet), wide area network (e.g. Internet), wireless LAN network (e.g., via Wi-Fi), cellular network, a SATCOM network, VHF network, a HF network, a WiMAX based network, and/or any other suitable communications network (or combination thereof) for transmitting data to and/or from the vehicle 108.

Each of the one or more remote computing devices 106 can include one or more processors and one or more memory devices. The one or more memory devices can be used to store instructions that when executed by the one or more processors of the one or more remote computing devices 106 cause the one or more processors to perform operations and/or functions including operations and/or functions associated with the vehicle 108 including exchanging (e.g., sending and/or receiving) data or signals with the vehicle 108, monitoring the state of the vehicle 108, and/or controlling the vehicle 108. The one or more remote computing devices 106 can communicate (e.g., exchange data and/or signals) with one or more devices including the operations computing system 104 and the vehicle 108 via the communications network 102. For example, the one or more remote computing devices 106 can request the location of the vehicle 108 via the communications network 102.

The one or more remote computing devices 106 can include one or more computing devices (e.g., a desktop computing device, a laptop computing device, a smart phone, and/or a tablet computing device) that can receive input or instructions from a user or exchange signals or data with an item or other computing device or computing system (e.g., the operations computing system 104). Further, the one or more remote computing devices 106 can be used to determine and/or modify one or more states of the vehicle 108 including a location (e.g., a latitude and longitude), a velocity, acceleration, a trajectory, and/or a path of the vehicle 108 based in part on signals or data exchanged with the vehicle 108. In some implementations, the operations computing system 104 can include the one or more remote computing devices 106.

The vehicle 108 can be a ground-based vehicle (e.g., an automobile), an aircraft, and/or another type of vehicle. The vehicle 108 can be an autonomous vehicle that can perform various actions including driving, navigating, and/or operating, with minimal and/or no interaction from a human driver. The autonomous vehicle 108 can be configured to operate in one or more modes including, for example, a fully autonomous operational mode, a semi-autonomous operational mode, a park mode, and/or a sleep mode. A fully autonomous (e.g., self-driving) operational mode can be one in which the vehicle 108 can provide driving and navigational operation with minimal and/or no interaction from a human driver present in the vehicle. A semi-autonomous operational mode can be one in which the vehicle 108 can operate with some interaction from a human driver present in the vehicle. Park and/or sleep modes can be used between operational modes while the vehicle 108 performs various actions including waiting to provide a subsequent vehicle service, and/or recharging between operational modes.

Furthermore, the vehicle 108 can include the one or more passenger compartment sensors 110 which can include one or more devices that can detect and/or determine one or more states of one or more objects inside the vehicle including one or more passengers. The one or more passenger compartment sensors 110 can be based in part on different types of sensing technology and can be configured to detect one or more biometric or physiological states of one or more passengers inside the vehicle including heart rate, blood pressure, body temperature, and/or respiratory rate.

An indication, record, and/or other data indicative of the state of the vehicle 108, the state of one or more passengers of the vehicle 108, and/or the state of an environment external to the vehicle 108 including one or more objects (e.g., the physical dimensions and/or appearance of the one or more objects) can be stored locally in one or more memory devices of the vehicle 108. Furthermore, the vehicle 108 can provide data indicative of the state of the one or more objects (e.g., physical dimensions and/or appearance of the one or more objects) within a predefined distance of the vehicle 108 to the operations computing system 104, which can store an indication, record, and/or other data indicative of the state of the one or more objects within a predefined distance of the vehicle 108 in one or more memory devices associated with the operations computing system 104 (e.g., remote from the vehicle).

The vehicle 108 can include and/or be associated with the vehicle computing system 112. The vehicle computing system 112 can include one or more computing devices located onboard the vehicle 108. For example, the one or more computing devices of the vehicle computing system 112 can be located on and/or within the vehicle 108. The one or more computing devices of the vehicle computing system 112 can include various components for performing various operations and functions. For instance, the one or more computing devices of the vehicle computing system 112 can include one or more processors and one or more tangible non-transitory, computer readable media (e.g., memory devices). The one or more tangible non-transitory, computer readable media can store instructions that when executed by the one or more processors cause the vehicle 108 (e.g., its computing system, one or more processors, and other devices in the vehicle 108) to perform operations and/or functions, including those described herein for receiving sensor data (e.g., sensor data associated with one or more states of one or more passengers in a passenger compartment of the vehicle 108) and/or vehicle data (e.g., vehicle data associated with one or more states of the vehicle 108) from sensors (e.g., the one or more sensors 114) of a vehicle (e.g., the vehicle 108); determining, based at least in part on the sensor data and/or the vehicle data, when the states of the passengers are associated with one or more health conditions; determining actions for the vehicle to perform based at least in part on the one or more health conditions associated with the passengers; and generating one or more control signals to control performance of the one or more actions by the vehicle. Furthermore, in some embodiments, the vehicle computing system 112 can perform one or more operations including accessing one or more machine-learned models (e.g., machine-learned models that are part of the vehicle computing system 112 and/or a remote computing device) that include one or more features of the one or more machine-learned models 1430 depicted in FIG. 14, the one or more machine-learned models 1470 which are depicted in FIG. 14, and/or the machine-learned passenger state model described in the method 1200 depicted in FIG. 12.

As depicted in FIG. 1, the vehicle computing system 112 can include the one or more sensors 114; the positioning system 118; the autonomy computing system 120; the communication system 136; the vehicle control system 138; and the human-machine interface 140. One or more of these systems can be configured to communicate with one another via a communication channel. The communication channel can include one or more data buses (e.g., controller area network (CAN)), on-board diagnostics connector (e.g., OBD-II), and/or a combination of wired and/or wireless communication links. The onboard systems can exchange (e.g., send and/or receive) data, messages, and/or signals amongst one another via the communication channel.

The one or more sensors 114 can be configured to generate and/or store data including the sensor data 116 associated with one or more objects that are proximate to the vehicle 108 (e.g., within range or a field of view of one or more of the one or more sensors 114). The one or more sensors 114 can include a Light Detection and Ranging (LIDAR) system, a Radio Detection and Ranging (RADAR) system, one or more cameras (e.g., visible spectrum cameras and/or infrared cameras), motion sensors, and/or other types of imaging capture devices and/or sensors. The sensor data 116 can include image data, radar data, LIDAR data, and/or other data acquired by the one or more sensors 114. The one or more objects can include, for example, pedestrians, vehicles, bicycles, and/or other objects. The one or more objects can be located on various parts of the vehicle 108 including a front side, rear side, left side, right side, top, or bottom of the vehicle 108. The sensor data 116 can be indicative of locations associated with the one or more objects within the surrounding environment of the vehicle 108 at one or more times. For example, sensor data 116 can be indicative of one or more LIDAR point clouds associated with the one or more objects within the surrounding environment. The one or more sensors 114 can provide the sensor data 116 to the autonomy computing system 120.

In addition to the sensor data 116, the autonomy computing system 120 can retrieve or otherwise obtain data including the map data 122. The map data 122 can provide detailed information about the surrounding environment of the vehicle 108. For example, the map data 122 can provide information regarding: the identity and location of different roadways, road segments, buildings, or other items or objects (e.g., lampposts, crosswalks and/or curb); the location and directions of traffic lanes (e.g., the location and direction of a parking lane, a turning lane, a bicycle lane, or other lanes within a particular roadway or other travel way and/or one or more boundary markings associated therewith); traffic control data (e.g., the location and instructions of signage, traffic lights, or other traffic control devices); and/or any other map data that provides information that assists the vehicle computing system 112 in processing, analyzing, and perceiving its surrounding environment and its relationship thereto.

The vehicle computing system 112 can include a positioning system 118. The positioning system 118 can determine a current position of the vehicle 108. The positioning system 118 can be any device or circuitry for analyzing the position of the vehicle 108. For example, the positioning system 118 can determine position by using one or more of inertial sensors, a satellite positioning system, based on IP/MAC address, by using triangulation and/or proximity to network access points or other network components (e.g., cellular towers and/or Wi-Fi access points) and/or other suitable techniques. The position of the vehicle 108 can be used by various systems of the vehicle computing system 112 and/or provided to one or more remote computing devices (e.g., the operations computing system 104 and/or the remote computing device 106). For example, the map data 122 can provide the vehicle 108 relative positions of the surrounding environment of the vehicle 108. The vehicle 108 can identify its position within the surrounding environment (e.g., across six axes) based at least in part on the data described herein. For example, the vehicle 108 can process the sensor data 116 (e.g., LIDAR data, camera data) to match it to a map of the surrounding environment to get a determination of the vehicle's position within that environment (e.g., transpose the vehicle's position within its surrounding environment).

The autonomy computing system 120 can include a perception system 124, a prediction system 126, a motion planning system 128, and/or other systems that cooperate to perceive the surrounding environment of the vehicle 108 and determine a motion plan for controlling the motion of the vehicle 108 accordingly. For example, the autonomy computing system 120 can receive the sensor data 116 from the one or more sensors 114, attempt to determine the state of the surrounding environment by performing various processing techniques on the sensor data 116 (and/or other data), and generate an appropriate motion plan through the surrounding environment. The autonomy computing system 120 can control the one or more vehicle control systems 138 to operate the vehicle 108 according to the motion plan.

The autonomy computing system 120 can identify one or more objects that are proximate to the vehicle 108 based at least in part on the sensor data 116 and/or the map data 122. For example, the perception system 124 can obtain state data 130 descriptive of a current and/or past state of an object that is proximate to the vehicle 108. The state data 130 for each object can describe, for example, an estimate of the object's current and/or past: location and/or position; speed; velocity; acceleration; heading; orientation; size/footprint (e.g., as represented by a bounding shape); class (e.g., pedestrian class vs. vehicle class vs. bicycle class), and/or other state information. The perception system 124 can provide the state data 130 to the prediction system 126 (e.g., for predicting the movement of an object).

The prediction system 126 can generate prediction data 132 associated with each of the respective one or more objects proximate to the vehicle 108. The prediction data 132 can be indicative of one or more predicted future locations of each respective object. The prediction data 132 can be indicative of a predicted path (e.g., predicted trajectory) of at least one object within the surrounding environment of the vehicle 108. For example, the predicted path (e.g., trajectory) can indicate a path along which the respective object is predicted to travel over time (and/or the velocity at which the object is predicted to travel along the predicted path). The prediction system 126 can provide the prediction data 132 associated with the one or more objects to the motion planning system 128.

The motion planning system 128 can determine a motion plan and generate motion plan data 134 for the vehicle 108 based at least in part on the prediction data 132 (and/or other data). The motion plan data 134 can include vehicle actions with respect to the objects proximate to the vehicle 108 as well as the predicted movements. For instance, the motion planning system 128 can implement an optimization algorithm that considers cost data associated with a vehicle action as well as other objective functions (e.g., cost functions based on speed limits, traffic lights, and/or other aspects of the environment), if any, to determine optimized variables that make up the motion plan data 134. By way of example, the motion planning system 128 can determine that the vehicle 108 can perform a certain action (e.g., pass an object) without increasing the potential risk to the vehicle 108 and/or violating any traffic laws (e.g., speed limits, lane boundaries, signage). The motion plan data 134 can include a planned trajectory, velocity, acceleration, and/or other actions of the vehicle 108.

The motion planning system 128 can provide the motion plan data 134 with data indicative of the vehicle actions, a planned trajectory, and/or other operating parameters to the vehicle control systems 138 to implement the motion plan data 134 for the vehicle 108. For instance, the vehicle 108 can include a mobility controller configured to translate the motion plan data 134 into instructions. By way of example, the mobility controller can translate a determined motion plan data 134 into instructions for controlling the vehicle 108 including adjusting the steering of the vehicle 108 “X” degrees and/or applying a certain magnitude of braking force. The mobility controller can send one or more control signals to the responsible vehicle control component (e.g., braking control system, steering control system and/or acceleration control system) to execute the instructions and implement the motion plan data 134.

The vehicle computing system 112 can include a communications system 136 configured to allow the vehicle computing system 112 (and its one or more computing devices) to communicate with other computing devices. The vehicle computing system 112 can use the communications system 136 to communicate with the operations computing system 104 and/or one or more other remote computing devices (e.g., the one or more remote computing devices 106) over one or more networks (e.g., via one or more wireless signal connections). In some implementations, the communications system 136 can allow communication among one or more of the system on-board the vehicle 108. The communications system 136 can also be configured to enable the autonomous vehicle to communicate with and/or provide and/or receive data and/or signals from a remote computing device 106 associated with a user and/or an item (e.g., an item to be picked-up for a courier service). The communications system 136 can utilize various communication technologies including, for example, radio frequency signaling and/or Bluetooth low energy protocol. The communications system 136 can include any suitable components for interfacing with one or more networks, including, for example, one or more: transmitters, receivers, ports, controllers, antennas, and/or other suitable components that can help facilitate communication. In some implementations, the communications system 136 can include a plurality of components (e.g., antennas, transmitters, and/or receivers) that allow it to implement and utilize multiple-input, multiple-output (MIMO) technology and communication techniques.

The vehicle computing system 112 can include the one or more human-machine interfaces 140. For example, the vehicle computing system 112 can include one or more display devices located on the vehicle computing system 112. A display device (e.g., screen of a tablet, laptop and/or smartphone) can be viewable by a user of the vehicle 108 that is located in the front of the vehicle 108 (e.g., driver's seat, front passenger seat). Additionally, or alternatively, a display device can be viewable by a user of the vehicle 108 that is located in the rear of the vehicle 108 (e.g., a back passenger seat). For example, the autonomy computing system 120 can provide one or more outputs including a graphical display of the location of the vehicle 108 on a map of a geographical area within one kilometer of the vehicle 108 including the locations of objects around the vehicle 108. A passenger of the vehicle 108 can interact with the one or more human-machine interfaces 140 by touching a touchscreen display device associated with the one or more human-machine interfaces to indicate, for example, a stopping location for the vehicle 108.

In some embodiments, the vehicle computing system 112 can activate, based at least in part on the sensor data, the vehicle data, and/or one or more health conditions determined by the vehicle computing system 112, one or more vehicle systems associated with operation of the vehicle 108. For example, the vehicle computing system 112 can send one or more control signals to activate one or more vehicle systems that can be used to send one or more notifications to a medical service provider and/or emergency medical contact, provide a passenger state query to a passenger (e.g., an audio or visual query requesting the subjective state of the passenger), and/or change the path of the vehicle 108 (e.g., sending one or more signals to an engine system and steering system of the vehicle). By way of further example, the vehicle computing system 112 can activate one or more vehicle systems including the communications system 136 that can send and/or receive signals and/or data with other vehicle systems, other vehicles, or remote computing devices (e.g., remote server devices); one or more lighting systems (e.g., one or more headlights, hazard lights, and/or vehicle compartment lights); one or more vehicle safety systems (e.g., one or more seatbelt or airbag systems); one or more notification systems that can generate one or more notifications for passengers of the vehicle 108 (e.g., auditory and/or visual messages about the state or predicted state of objects external to the vehicle 108 including the location of emergency medical service vehicles or personnel); braking systems; propulsion systems that can be used to change the acceleration and/or velocity of the vehicle; and/or steering systems that can change the path, course, and/or direction of travel of the vehicle 108.

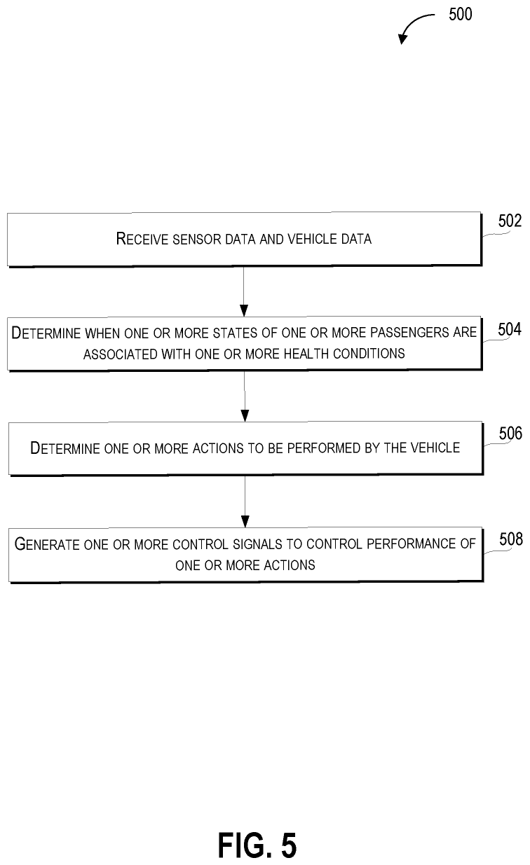

In some embodiments, the vehicle computing system 112 can perform one or more operations including sending sensor data and/or the vehicle data to a machine-learned passenger state model (e.g., the machine-learned model 1430 and/or the machine-learned model 1470 depicted in FIG. 14) trained to receive the sensor data and the vehicle data, and provide an output including one or more health condition predictions associated with one or more health conditions (e.g., the one or more health conditions described in the method 500 depicted in FIG. 5); and receiving, from the machine-learned passenger state model, the output including the one or more health condition predictions associated with the one or more health conditions.

In some embodiments, the vehicle computing system 112 can perform one or more operations including determining a respiratory pattern for each of one or more passengers of the vehicle 108 based at least in part on one or more changes in air pressure inside a passenger compartment of the vehicle 108 or one or more sensor outputs of the one or more sensors 114 including a motion sensing respiration sensor in a safety restraint device of the vehicle 108; and comparing the respiratory pattern for each of the one or more passengers to a set of respiratory rates associated with the one or more health conditions (e.g., the one or more health conditions described in the method 500 depicted in FIG. 5). For example, the vehicle computing system 112 can compare a respiratory pattern of each of one or more passengers to respiratory pattern data that includes various respiratory rates (e.g., determining whether the respiratory rate associated with a respiratory pattern is lower, higher, or within a range associated with a respiratory rate threshold), and respiratory volumes (e.g., the amount of air inhaled and/or an amount of carbon dioxide exhaled).

In some embodiments, the vehicle computing system 112 can perform one or more operations including determining a location of the vehicle 108; and sending one or more notifications to an emergency medical provider (e.g., one or more notifications including the location of the vehicle 108 and the one or more states of the one or more passengers).

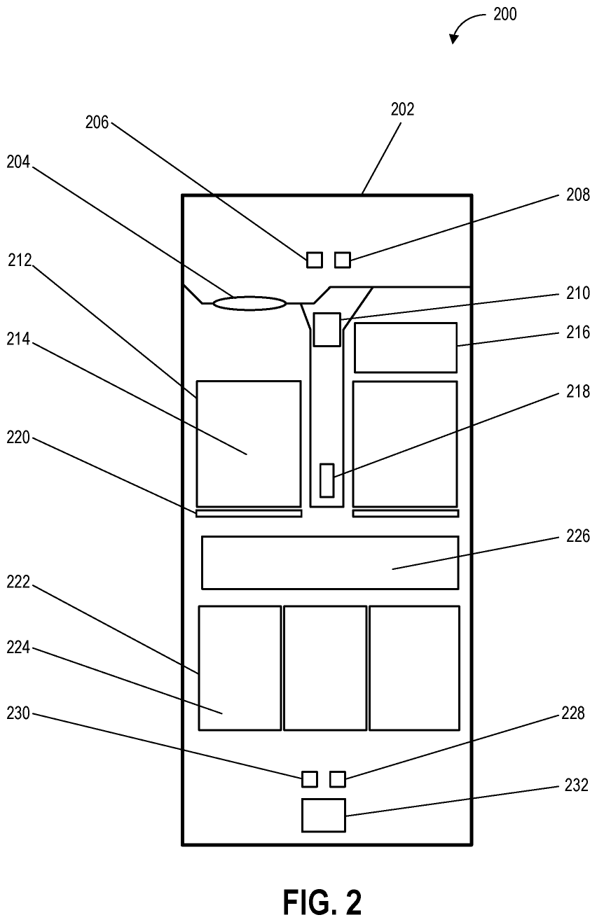

FIG. 2 depicts an example of an environment including a passenger compartment of an autonomous vehicle according to example embodiments of the present disclosure. One or more functions or operations performed in FIG. 2 can be implemented or performed by one or more devices (e.g., one or more computing devices) or systems including, for example, the operations computing system 104, the vehicle 108, or the vehicle computing system 112, shown in FIG. 1. As illustrated, FIG. 2 shows a vehicle 200 (e.g., a vehicle including one or more features of the vehicle 108 depicted in FIG. 1) that includes a passenger compartment 202, a steering control sensor 204, a front camera 206, a front microphone 208, a front information device 210, a front seat 212, a front seat sensor 214, a front floor sensor 216, a passenger compartment pressure sensor 218, a rear information device 220, a rear seat 222, a rear seat sensor 224, a rear floor sensor 226, a rear camera 228, a rear microphone 230, and a vehicle computing system 232.

The vehicle 200 includes a passenger compartment 202 that can accommodate one or more passengers and/or cargo. The passenger compartment 202 can include various sensors that can be used to determine the state of one or more passengers within the passenger compartment 202. Further, the passenger compartment 202 can include one or more doors and/or windows that can be opened and/or closed, and through which one or more passengers and/or cargo can gain entry to, or exit, the passenger compartment 202. The steering control sensor 204 can include one or more tactile sensors and/or one or more thermal sensors that are part of a steering control mechanism (e.g., a steering wheel) of the vehicle 200 and that can be used to determine various physiological characteristics of a passenger including the passenger's grip strength, skin temperature, blood pressure, and/or heart rate (e.g., determining heart rate based on electrical current detected on a passenger's skin surface).

The passenger compartment 202 can include the front camera 206 and the rear camera 228 that can be used to capture one or more images of one or more passengers within the passenger compartment 202. The front camera 206 and/or the rear camera 228 can include one or more thermal cameras, one or more infrared cameras, and/or one or more visual spectrum cameras that can be used by the vehicle computing system 232 (e.g., a computing system that includes one or more features of the vehicle computing system 112) to determine one or more states and/or one or more health conditions of one or more passengers in the passenger compartment 202. Further, the front camera 206 and/or rear camera 228 can be used to capture one or more images of one or more passengers of the passenger compartment 202. In some embodiments, the one or more images captured by the front camera 206 and/or the rear camera 228 can be used to determine various characteristics of a passenger including motion characteristics of a passenger, body temperature of a passenger, and/or body position of a passenger (e.g., whether a passenger is sitting up straight or slumping). Further, the vehicle computing system 232 can receive sensor data associated with the one or more images captured by the front camera 206 and/or the rear camera 228. The sensor data can be used by the vehicle computing system 232 to determine one or more states and/or one or more health conditions of one or more passengers of the vehicle 200.

The front seat 212 and the rear seat 222 can be configured to accommodate passengers or cargo and can include the front seat sensor 214 and the rear seat sensor 224 respectively. The front seat sensor 214 and the rear seat sensor 224 can include one or more tactile and/or pressure sensors that can be used to detect one or more motions of a passenger seated on the front seat sensor 214 and/or the rear seat sensor 224. For example, the front seat sensor 214 and/or the rear seat sensor 224 can generate sensor data based on the mass or weight of a passenger detected by the front seat sensor and/or the rear seat sensor 224, a direction of passenger movement detected by the front seat sensor 214 and/or the rear seat sensor 224, the distribution of a passenger's weight across the front seat sensor 214 and/or the rear seat sensor 224, and/or the velocity of a passenger's movement detected by front seat sensor 214 and/or the rear seat sensor 224. The sensor data generated by the front seat sensor 214 and/or the rear seat sensor 224 can be used by the vehicle computing system 232 to determine one or more states and/or one or more health conditions of one or more passengers of the vehicle 200.

The vehicle compartment 202 can include the front microphone 208 and/or the rear microphone 230 that can detect one or more sounds including one or more sounds produced by one or more passengers of the vehicle 200. The front microphone 208 and/or the rear microphone 230 can generate sensor data that can be used by the vehicle computing system 232 to determine one or more states and/or one or more health conditions of one or more passengers of the vehicle 200.

The vehicle compartment 202 can include the passenger compartment pressure sensor 218 that can detect air pressure and/or changes in the air pressure inside the passenger compartment 202. The changes in the air pressure of the passenger compartment 202 detected by the passenger compartment pressure sensor 218 can be used to determine one or more characteristics of one or more passengers of the vehicle 200 including a respiratory pattern of one or more passengers (e.g., rate of respiration). The passenger compartment pressure sensor 218 can generate sensor data that can be used by the vehicle computing system 232 to determine one or more states and/or one or more health conditions of one or more passengers of the vehicle 200.

The vehicle compartment 202 can include the front floor sensor 216 and/or rear floor sensor 226 that can detect changes in the pressure exerted on the front floor sensor 216 and/or the rear floor sensor 226 inside the passenger compartment 202 (e.g., the pressure exerted by the feet of one or more passengers of the vehicle 200). The changes in pressure detected by the front floor sensor 216 and/or rear floor sensor 226 can be used to generate sensor data that can be used by the vehicle computing system 232 to determine one or more characteristics of one or more passengers of the vehicle 200 including the rate and intensity of movements made by the feet of the one or more passengers. Further, the front floor sensor 216 and/or rear floor sensor 226 can generate sensor data that can be used by the vehicle computing system 232 to determine one or more states and/or one or more health conditions of one or more passengers of the vehicle 200.

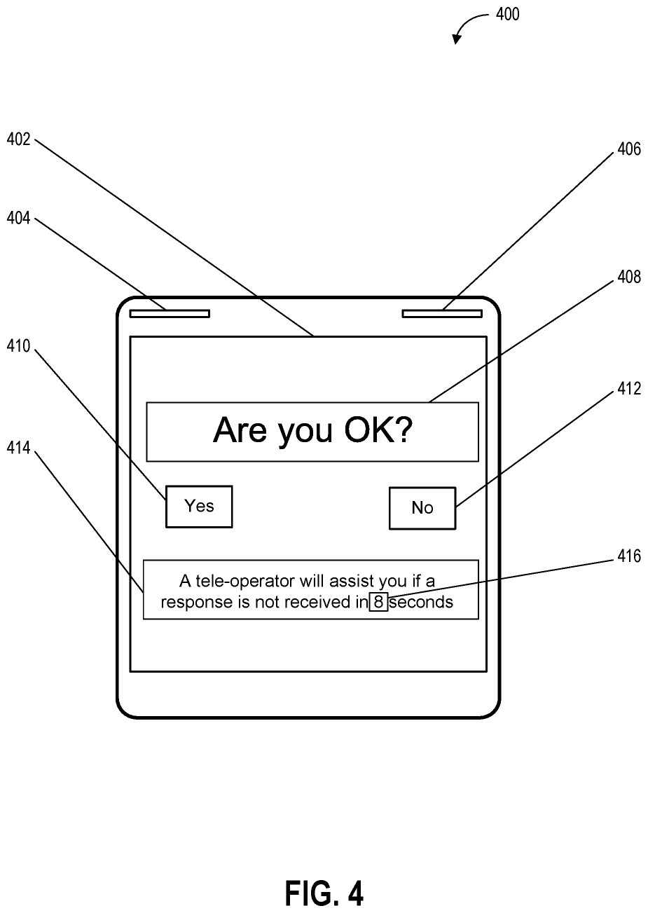

The vehicle compartment 202 can include one or more display devices including the front information device 210 and/or the rear information device 220 that can be used to provide information and/or receive feedback from one or more passengers of the vehicle 200. For example, the front information device 210 and/or the rear information device 220 can provide a passenger state query requesting the state of a passenger (e.g., asking a passenger if the passenger is feeling well). The front information device 210 and/or the rear information device 220 can, for example, include one or more features of the computing device 400 depicted in FIG. 4. Further, the front information device 210 and/or the rear information device 220 can send and/or receive data to and from the vehicle computing system 232.

In some embodiments, one or more sensor outputs of the steering control sensor 204, the front camera 206, the front microphone 208, the front seat sensor 214, the front floor sensor 216, the passenger compartment pressure sensor 218, the rear seat 222, the rear seat sensor 224, the rear floor sensor 226, the rear camera 228, and/or the rear microphone 230 can be include in the sensor data described in the method 500 depicted in FIG. 5.

In some embodiments, the vehicle computing system 232 can perform one or more operations including receiving sensor data (e.g., sensor data associated with one or more states of one or more passengers in a passenger compartment of the vehicle 108 depicted in FIG. 1) and/or vehicle data (e.g., vehicle data associated with one or more states of the vehicle 108 depicted in FIG. 1) from sensors (e.g., the one or more sensors 114 depicted in FIG. 1) of a vehicle (e.g., the vehicle 108 depicted in FIG. 1); determining, based at least in part on the sensor data and/or the vehicle data, when the states of the passengers are associated with one or more health conditions as described in the method 500 depicted in FIG. 5; determining actions for the vehicle to perform based at least in part on the one or more health conditions associated with the passengers as described in the method 500 depicted in FIG. 5; and generating one or more control signals to control performance of the one or more actions by the vehicle as described in the method 500 depicted in FIG. 5. Furthermore, in some embodiments, the vehicle computing system 232 can perform one or more operations including accessing one or more machine-learned models that include one or more features of the one or more machine-learned models 1430 and/or the one or more machine-learned models 1470 which are depicted in FIG. 14.

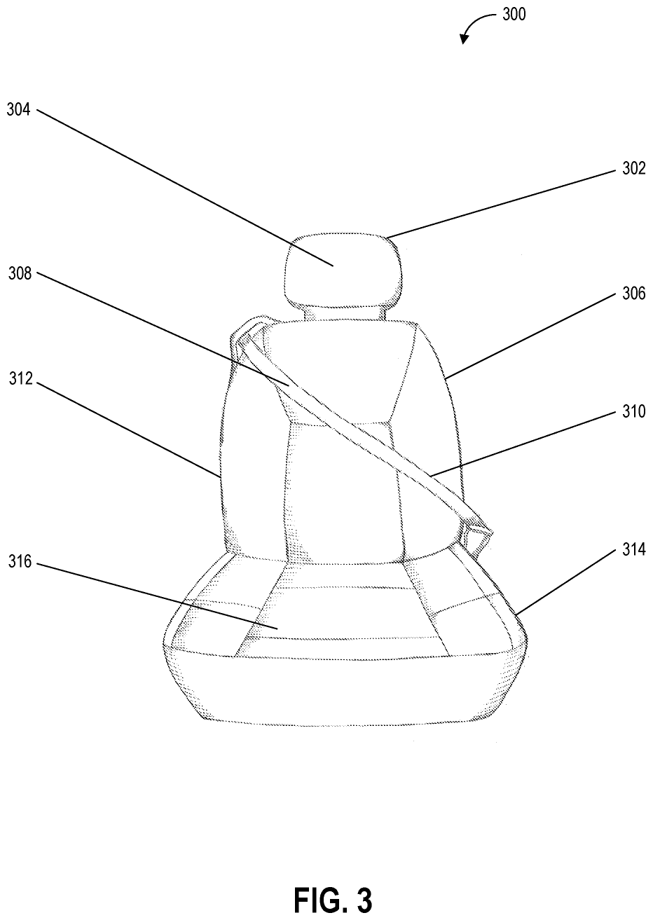

FIG. 3 depicts an example of passenger seating area sensors according to example embodiments of the present disclosure. One or more actions or events depicted in FIG. 3 can be implemented by one or more devices (e.g., one or more computing devices) or systems including, for example, the operations computing system 104, the vehicle 108, or the vehicle computing system 112, which are shown in FIG. 1. FIG. 3 includes an illustration of a passenger seating device 300 that can be used to exchange (e.g., send and/or receive) one or more signals or data with one or more computing systems including, for example, the operations computing system 104, the vehicle 108, or the vehicle computing system 112, which are shown in FIG. 1. As shown, FIG. 3 illustrates the passenger seating device 300, a headrest 302, a headrest sensor 304, a seatbelt 306, one or more seatbelt sensors 308, an upper seating area 310, one or more upper seating area sensors 312, a lower seating area 314, and one or more lower seating area sensors 316.

The passenger seating device 300 can include the headrest 302 that can include one or more sensors including the headrest sensor 304. The headrest sensor 304 can include one or more tactile and/or pressure sensors that can detect physical contact by a portion of a passenger's body including the passenger's head and/or neck. For example, the headrest sensor 304 can determine the rate and/or pattern of movement of a passenger's head against the headrest sensor 304. Based at least in part on the rate and pattern of movement of a passenger's head against the headrest sensor 304, the headrest sensor 304 can generate sensor data that can be used by a computing system (e.g., the vehicle computing system 112 depicted in FIG. 1) to determine whether the passenger's state is associated with one or more health conditions. For example, the vehicle computing system 112 can determine that a passenger is experiencing a seizure or has lost consciousness based at least in part on the pressure of the passenger's head on the headrest sensor 304.

The seatbelt 306 can include one or more seatbelt sensors 308 can detect and/or determine various states of a passenger including one or more biometric or physiological states (e.g., heart rate and/or blood pressure). The one or more seatbelt sensors 308 can include one or more tactile, electrostatic, capacitive, and/or pressure sensors that can be used to determine the one or more biometric and/or physiological states of a passenger seated in the passenger seating device 300. For example, the one or more seatbelt sensors 308 can be used to determine a respiratory pattern (e.g., respiratory rate) and/or a heart rate of a passenger occupying the passenger seating device 300. Further, the one or more seatbelt sensors 308 can generate sensor data that can be used by a computing system (e.g., the vehicle computing system 112 depicted in FIG. 1 and/or the vehicle computing system 232 depicted in FIG. 2) to determine one or more states and/or one or more health conditions of a passenger occupying the passenger seating device 300.

The upper seating area 310 and the lower seating area 314 can include the one or more upper seating area sensors 312 and the one or more lower seating area sensors 316 respectively. The one or more upper seating area sensors 312 and the one or more lower seating area sensors 316 can detect and/or determine various states of a passenger including one or more physical characteristics (e.g., body mass, body weight, and/or height), biometric states, and/or physiological states (e.g., heart rate and/or blood pressure). The one or more upper seating area sensors 312 and the one or more lower seating area sensors 316 can include one or more tactile, electrostatic, capacitive, strain gauges, and/or pressure sensors that can be used to determine the one or more physical characteristics, biometric states, and/or physiological states of a passenger seated in the passenger seating device 300. For example, the one or more upper seating area sensors 312 and the one or more lower seating area sensors 316 can be used to determine the weight and/or mass (e.g., determine mass using sensors in the one or more lower seating area sensors 316) and height (e.g., determine height based on the pressure exerted on portions of the one or more upper seating area sensors 312) of a passenger occupying the passenger seating device 300. Further, the one or more upper seating area sensors 312 and the one or more lower seating area sensors 316 can generate sensor data that can be used by a computing system (e.g., the vehicle computing system 112 depicted in FIG. 1 and/or the vehicle computing system 232 depicted in FIG. 2) to determine one or more states and/or one or more health conditions of a passenger occupying the passenger seating device 300.

FIG. 4 depicts an example of a computing system including a display device according to example embodiments of the present disclosure. One or more actions or events depicted in FIG. 4 can be implemented by one or more devices (e.g., one or more computing devices) or systems including, for example, the operations computing system 104, the vehicle 108, or the vehicle computing system 112, shown in FIG. 1. FIG. 4 includes an illustration of a computing device 400 that can be used exchange (e.g., send and/or receive) one or more signals or data with one or more computing systems including, for example, the operations computing system 104, the vehicle 108, or the vehicle computing system 112, which are shown in FIG. 1. As shown, FIG. 4 illustrates the computing device 400, a display component 402, a speaker component 404, a microphone component 406, a passenger state query element 408, a feedback control element 410, a feedback control element 412, a notification element 414, and a time indicator element 416.

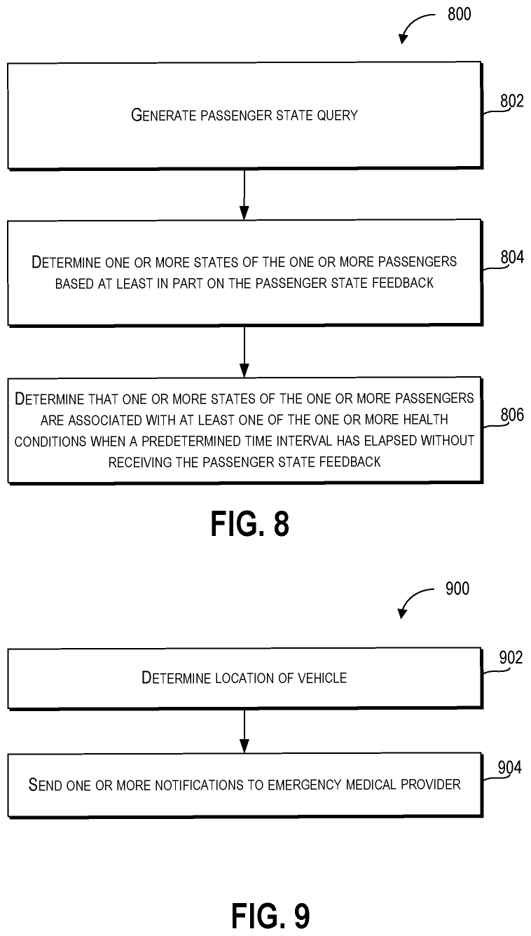

The computing device 400 (e.g., a computing device with one or more features of the vehicle computing system 112 depicted in FIG. 1 and/or the vehicle computing system 232 depicted in FIG. 2) includes a display component 402 (e.g., a display component that can include one or more features of the front information device 210 and/or the rear information device 220 which are depicted in FIG. 2) that can be used to display information including the passenger state query element 408 and the notification element 414. In this example, the display component 402 is a touch screen display that is configured to detect tactile interactions with the display component 402 (e.g., a passenger touching the display component 402). Further, the computing device 400 can generate audio output including information associated with the passenger state query element 408 (e.g., a passenger state query element including one or more features of the passenger state query described in the method 800 depicted in FIG. 8) and the notification element 414 via the speaker component 404.

In this example, the passenger state query element 408 includes a feedback query (“Are you OK?”) that is directed to a passenger of a vehicle (e.g., the vehicle 108). The passenger can respond by touching the feedback control element 410 (“Yes”) to indicate that the passenger is feeling OK (e.g., feeling well) or by touching the feedback control element 412 (“No”) to indicate that the passenger is not feeling well and that the passenger may be experiencing one or more health conditions. In this way, a passenger can provide a direct subjective assessment of the state of their own wellbeing that can be used to determine whether the passenger is experiencing one or more health conditions.

Further, the computing device 400 can generate the notification element 414 includes the message, “A tele-operator will assist you if a response is not received in 8 seconds”, that is directed to a passenger of a vehicle (e.g., the vehicle 108). The notification element 414 can include a time indicator element 416 that can countdown from a predetermined number of seconds (e.g., ten seconds) to zero seconds and indicate an amount of time available before a tele-operator is contacted.

In some embodiments, a passenger state query can be communicated via the speaker component 404. Further, a passenger can provide feedback by speaking to the microphone component 406 that can transmit a passenger vocalization to the computing device 400.

In some embodiments, the computing device 400 can perform one or more operations including receiving sensor data (e.g., sensor data associated with one or more states of one or more passengers in a passenger compartment of the vehicle 108 depicted in FIG. 1) and/or vehicle data (e.g., vehicle data associated with one or more states of the vehicle 108) from sensors (e.g., the one or more sensors 114 depicted in FIG. 1) of a vehicle (e.g., the vehicle 108 depicted in FIG. 1); determining, based at least in part on the sensor data and/or the vehicle data, when the states of the passengers are associated with one or more health conditions; determining actions for the vehicle to perform based at least in part on the one or more health conditions associated with the passengers; and generating one or more control signals to control performance of the one or more actions by the vehicle. Furthermore, in some embodiments, the computing device 400 can perform one or more operations including accessing one or more machine-learned models that include one or more features of the one or more machine-learned models 1430 and/or the one or more machine-learned models 1470 which are depicted in FIG. 14.

FIG. 5 depicts a flow diagram of an example method of passenger monitoring and intervention according to example embodiments of the present disclosure. One or more portions of a method 500 can be implemented by one or more devices (e.g., one or more computing devices) or systems including, for example, the operations computing system 104, the vehicle 108, or the vehicle computing system 112, shown in FIG. 1. Moreover, one or more portions of the method 500 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 5 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 502, the method 500 can include receiving sensor data and vehicle data from one or more sensors of a vehicle. The sensor data can be associated with one or more states of one or more passengers in a passenger compartment of the vehicle. The vehicle data can be associated with one or more states of the vehicle. For example, the vehicle (e.g., the vehicle 108) and or a computing device and/or computing system associated with the vehicle (e.g., the vehicle computing system 112) can include one or more transmitters and/or receivers that can send and/or receive one or more signals (e.g., one or more signals that are transmitted and/or received wirelessly and/or via a wired connection) including the sensor data and/or the vehicle data.

In some embodiments, the vehicle data can include information associated with a velocity of the vehicle (e.g., a velocity in meters per second), an acceleration of the vehicle (e.g., a rate at which the vehicle accelerates), a deceleration of the autonomous vehicle (e.g., a rate at which the vehicle decelerates), a centrifugal force on the passenger compartment of the vehicle, a temperature of the passenger compartment of the vehicle, a pressure in the passenger compartment of the vehicle (e.g., a barometric pressure), and/or a humidity of the passenger compartment of the vehicle.

In some embodiments, the one or more sensors can include one or more heart rate sensors (e.g., an electrocardiogram device), one or more respiratory rate sensors, one or more image sensors, one or more microphones, and/or one or more thermal sensors. Further, the one or more sensors can include various other biometric devices that can be used to measure one or more states of a person (e.g., a passenger of a vehicle) including blood pressure, galvanic skin response, perspiration, and/or blood alcohol content.

At 504, the method 500 can include determining, based at least in part on the sensor data and/or the vehicle data, when the one or more states of the one or more passengers are associated with one or more health conditions. For example, the vehicle computing system 112 can compare the sensor data and the vehicle data to a set of thresholds associated with one or more physical characteristics. In particular, the heart rate of a passenger can be determined based on one or more sensor outputs from the one or more sensors 114 (e.g., a heart rate monitor) of the vehicle 108. The vehicle computing system 112 can then compare the heart rate of the passenger to a set of heart rate ranges associated with one or more health conditions. When combined with other determined states of the passenger (e.g., passenger movement patterns, passenger respiratory rate, and/or passenger blood pressure) the heart rate of the passenger exceeding a certain heart rate threshold can be associated with one or more health conditions (e.g., paroxysmal supraventricular tachycardia).

In some embodiments, the one or more health conditions can include a dyspneic state, a state of emesis, a seizure state, a cardiac arrest state, and/or a stroke state. Furthermore, the one or more health conditions can include any health condition and/or medical condition associated with the state of a person (e.g., the state of a passenger of the vehicle).

Furthermore, the one or more health conditions can include any health condition that partly or completely reduces and/or interferes with the one or more passengers ability to self-report their health condition. For example, a passenger that is experiencing a seizure or cardiac arrest may be unable to speak clearly (or unable to speak at all) and may also have impairment of their motor abilities or motor skills (e.g., the ability to control hand movements including gestures and/or interactions with a user interface) that prevent the passenger from indicating that the passenger is experiencing a health condition. Under such circumstances or similar circumstances, the vehicle computing system 112 can determine (e.g., distinguish) the one or more health conditions in which a passenger is able to self-report their health condition (e.g., provide a verbal indication, gesture, or input to another passenger or an input device that the passenger is experiencing a health condition) and the one or more health conditions in which a passenger is unable to self-report their health condition. Based on the determination that a passenger is unable to self-report their health condition (e.g., a passenger not responding to a passenger state query or providing improper feedback to a passenger state query as described in FIG. 4) or unable to treat their own health condition, the vehicle computing system 112 can generate different control signals to perform different types of actions. For example, when the vehicle computing system 112 determines that a passenger is unable to self-report their health condition (e.g., the passenger is determined to have a health condition that is associated with unresponsiveness (e.g., stroke or cardiac arrest) and/or does not respond to a passenger state query as described in FIG. 4), the vehicle computing system 112 can perform a set of actions to address the health condition. The set of actions can include, for example, the vehicle computing system 112 generating one or more signals to guide and/or drive the vehicle 108 to a hospital, sending information describing the passenger's health condition to a hospital or emergency medical provider, and/or notifying a hospital or emergency medical provider that emergency services are needed at the location of the vehicle 108.

In some embodiments, determining, based at least in part on the sensor data and/or the vehicle data, when the one or more states of the one or more passengers are associated with one or more health conditions can include the use of a machine-learned model. The machine-learned model can operate in a portion of the vehicle computing system 112 and include one or more features of the one or more machine-learned models 1430 depicted in FIG. 14, the one or more machine-learned models 1470 depicted in FIG. 14, and/or the machine-learned passenger state model described in the method 1200 depicted in FIG. 12. For example, the machine-learned model 1470 can receive the sensor data and/or the vehicle data from the one or more sensors 114 of the vehicle 108 and provide an output (e.g., output data) including one or more state predictions associated with one or more states of the one or more passengers are associated with one or more health conditions.

At 506, the method 500 can include determining, based at least in part on the one or more health conditions associated with the one or more passengers, one or more actions to be performed by the vehicle. For example, the vehicle computing system 112 can determine that when the state of a passenger of the vehicle 108 is determined to be associated with a movement pattern corresponding to a seizure, that the vehicle will send a notification to an emergency medical contact associated with the passenger.

In some embodiments, determining, based at least in part on the one or more health conditions associated with the one or more passengers, one or more actions to be performed by the vehicle can include the use of a machine-learned model. The machine-learned model can operate in a portion of the vehicle computing system 112 and include one or more features of the one or more machine-learned models 1430 depicted in FIG. 14, the one or more machine-learned models 1470 depicted in FIG. 14, and/or the machine-learned passenger state model described in the method 1200 depicted in FIG. 12. For example, the machine-learned model 1470 can operate in a portion of the vehicle computing system 112 and can receive input data comprising information associated with the one or more health conditions associated with the one or more passengers and provide an output (e.g., output data) including one or more actions for the vehicle 108 to perform.

At 508, the method 500 can include generating, one or more control signals to control performance of the one or more actions by the vehicle. For example, the vehicle computing system 112 can send one or more control signals to a navigation device (e.g., a GPS) of the vehicle 108 that can be used to determine the location of the vehicle. Further, the vehicle computing system 112 can access local map data and/or send one or more signals to a geographic information service provider (e.g., a remote computing device that includes information associated with one or more maps of a geographic area) to determine the location of the nearest hospital to the vehicle 108. Using the location of the vehicle 108 and the location of the nearest hospital to the vehicle 108, the vehicle computing system 112 can generate one or more control signals to drive the vehicle 108 to the hospital.

In some embodiments, the one or more actions performed by the vehicle (e.g., the vehicle 108) can include sending one or more signals to a medical facility, sending one or more signals to a vehicle tele-operator, sending one or more notifications to an emergency medical contact associated with at least one of the one or more passengers, and/or driving the vehicle to a destination determined by at least one of the one or more passengers.

Furthermore, the one or more actions performed by the vehicle can include activating one or more vehicle systems including passenger compartment systems (e.g., opening vehicle windows to improve ventilation, increasing or reducing the temperature in the passenger compartment, and/or turning off music or other entertainment media that is playing in the vehicle); illumination systems (e.g., turning on hazard lights of the vehicle 108); notification systems (e.g., generate a textual message requesting assistance on a display portion of the vehicle 108 that is visible to an observer external to the vehicle 108); braking systems (e.g., reducing the sharpness of applying brakes when a passenger with a health condition is being conveyed to a medical facility); propulsion systems (e.g., reducing the rate of acceleration when a passenger with a health condition is being conveyed to a medical facility); and/or steering systems (e.g., steering the vehicle more gently when a passenger with a health condition is being conveyed to a hospital).

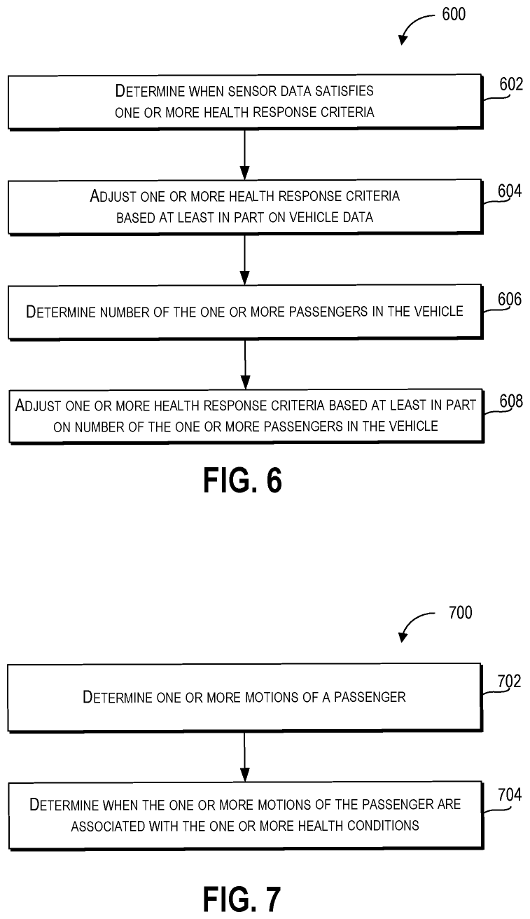

FIG. 6 depicts a flow diagram of an example method of passenger monitoring and intervention according to example embodiments of the present disclosure. One or more portions of a method 600 can be implemented by one or more devices (e.g., one or more computing devices) or systems including, for example, the operations computing system 104, the vehicle 108, or the vehicle computing system 112, shown in FIG. 1. Moreover, one or more portions of the method 600 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 6 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 602, the method 600 can include determining when the sensor data (e.g., the sensor data of the method 500) satisfies one or more health response criteria associated with an occurrence of the one or more health conditions (e.g., the one or more health conditions of the method 500). The one or more health response criteria can include one or more threshold levels or other quantitative or qualitative criteria associated with the one or more states of the one or more passengers (e.g., physiological states of the one or more passengers that are associated with one or more health conditions). For example, the one or more health response criteria can include a heart rate threshold or respiratory rate threshold that can be used to indicate the occurrence of a health condition. Furthermore, satisfying the one or more health response criteria can include a comparison (e.g., a comparison by the vehicle computing system 112) of one or more attributes, parameters, and/or values of the sensor data and/or vehicle data (e.g., the vehicle data of the method 500) to one or more corresponding attributes, parameters, and/or values of health response criteria data associated with the one or more health response criteria.

For example, the vehicle computing system 112 can determine that the one or more health response criteria are satisfied when the heart rate or blood pressure of a passenger of the vehicle 108 falls below a respective heart rate threshold or blood pressure threshold.

In some embodiments, determining when the sensor data satisfies one or more health response criteria associated with an occurrence of the one or more health conditions can be used in determining, based at least in part on the sensor data and the vehicle data, when the one or more states of the one or more passengers are associated with one or more health conditions as described in 504 of the method 500 depicted in FIG. 5.

At 604, the method 600 can include adjusting the one or more health response criteria based at least in part on the vehicle data. Adjusting (e.g., modifying and/or changing) the one or more health response criteria can include increasing and/or decreasing any values associated with the one or more health response criteria; and/or changing the one or more health response criteria that are used. For example, the vehicle computing system 112 can determine (based on the vehicle data) that the vehicle 108 is rounding a corner, causing a passenger of the vehicle to lean into the turn, thereby changing the passenger's position with respect to a seat of the vehicle 108. The vehicle computing system 112 can then adjust (e.g., change the sensitivity to passenger movement of one or more seat sensors) the one or more health response criteria associated with passenger movement based on the determination that the vehicle 108 is rounding the corner. By way of further example, the vehicle computing system 112 can determine, in response to detecting that the vehicle 108 is braking, adjust the one or more health response criteria associated with passenger movement against a safety restraint device (e.g., a seat belt) of the vehicle 108.

In some embodiments, adjusting the one or more health response criteria based at least in part on the vehicle data can be used in determining, based at least in part on the sensor data and the vehicle data, when the one or more states of the one or more passengers are associated with one or more health conditions as described in 504 of the method 500 depicted in FIG. 5.

At 606, the method 600 can include determining a number of the one or more passengers in the vehicle. For example, the vehicle computing system 112 can use the one or more sensors 114 (e.g., one or more cameras and/or one or more thermal sensors) to determine the number of passengers in the vehicle 108.

In some embodiments, determining a number of the one or more passengers in the vehicle can be used in determining, based at least in part on the sensor data and the vehicle data, when the one or more states of the one or more passengers are associated with one or more health conditions as described in 504 of the method 500 depicted in FIG. 5.

At 608, the method 600 can include adjusting the one or more health response criteria based at least in part on the number of the one or more passengers in the vehicle. For example, the vehicle computing system 112 can, after determining that a passenger compartment of the vehicle 108 contains three passengers, adjust one or more carbon dioxide threshold values that are part of the one or more health response criteria associated with passenger respiration (e.g., one or more carbon dioxide levels by one or more passengers that are associated with one or more health conditions).

In some embodiments, adjusting the one or more health response criteria based at least in part on the number of the one or more passengers in the vehicle can be used in determining, based at least in part on the sensor data and the vehicle data, when the one or more states of the one or more passengers are associated with one or more health conditions as described in 504 of the method 500 depicted in FIG. 5.

FIG. 7 depicts a flow diagram of an example method of passenger monitoring and intervention according to example embodiments of the present disclosure. One or more portions of a method 700 can be implemented by one or more devices (e.g., one or more computing devices) or systems including, for example, the operations computing system 104, the vehicle 108, or the vehicle computing system 112, shown in FIG. 1. Moreover, one or more portions of the method 700 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 7 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 702, the method 700 can include determining, based at least in part on the sensor data (e.g., the sensor data of the method 500), one or more characteristics of one or more motions of a passenger of the one or more passengers. In some embodiments, the one or more motions of a passenger can include one or more motions with respect to a seating area (e.g., a vehicle seat) of the passenger in the passenger compartment of the vehicle. The one or more characteristics of the one or more motions of the passenger can include a velocity of motion of any portion of a passenger's body, an acceleration of motion of any portion of a passenger's body, a range of motion of any portion of a passenger's body, and/or a direction of motion of any portion of a passenger's body. Further, the one or more characteristics of the one or more motions of the passenger can include one or more motion patterns (e.g., reciprocal arm and/or leg motions) that may be directly or partly associated with one or more health conditions.

By way of example, the vehicle computing system 112 can receive one or more sensor outputs from the one or more sensors 114 (e.g., one or more cameras, one or more seat pressure sensors, and/or one or more thermal sensors) that can be used to track one or more motions (e.g., track the movement of the head, face, eyes, arms, hands, legs, feet, and/or torso of the one or more passengers) of a passenger of the one or more passengers in the vehicle 108. Further, the one or more sensor outputs can be used by the vehicle computing system to determine one or more characteristics of the one or more motions of the passenger. By way of further example, the vehicle 108 can include motion sensors in a seating area that can detect the motions of a passenger including fidgeting movements of the passenger in the seating area.

In some embodiments, determining, based at least in part on the sensor data, one or more characteristics of one or more motions of a passenger of the one or more passengers can be used in determining, based at least in part on the sensor data and the vehicle data, when the one or more states of the one or more passengers are associated with one or more health conditions as described in 504 of the method 500 depicted in FIG. 5.

At 704, the method 700 can include determining when the one or more motions of the passenger satisfy one or more passenger motion criteria associated with the one or more health conditions. The one or more passenger motion criteria can include one or more threshold levels or other quantitative or qualitative criteria associated with the one or more states of the one or more passengers (e.g., types of motion by a passenger or motion by a passenger in certain portions of the vehicle). For example, the one or more passenger motion criteria can include one or more motion patterns associated with various health conditions (e.g., motion patterns associated with a seizure or cardiac arrest). Furthermore, satisfying the one or more passenger motion criteria can include a comparison (e.g., a comparison by the vehicle computing system 112) of one or more attributes, parameters, and/or values of the sensor data and/or vehicle data (e.g., the vehicle data of the method 500) to one or more corresponding attributes, parameters, and/or values of passenger motion criteria data associated with the one or more passenger motion criteria.

In some embodiments, determining when the one or more motions of the passenger satisfy one or more passenger motion criteria associated with the one or more health conditions can be used in determining, based at least in part on the sensor data and the vehicle data, when the one or more states of the one or more passengers are associated with one or more health conditions as described in 504 of the method 500 depicted in FIG. 5.

FIG. 8 depicts a flow diagram of an example method of passenger monitoring and intervention according to example embodiments of the present disclosure. One or more portions of a method 800 can be implemented by one or more devices (e.g., one or more computing devices) or systems including, for example, the operations computing system 104, the vehicle 108, or the vehicle computing system 112, shown in FIG. 1; or the computing system 1400 shown in FIG. 14. Moreover, one or more portions of the method 800 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 8 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 802, the method 800 can include generating a passenger state query including a request for passenger state feedback based at least in part on the one or more states of the one or more passengers (e.g., the one or more states of the one or more passengers in the method 500 depicted in FIG. 5). The passenger state query can include one or more visual indications and/or one or more auditory indications. For example, the vehicle computing system 112 can generate one or more control signals that activate a display device in the vehicle 108 (e.g., a display device including one or more features of the rear information device 220 depicted in FIG. 2 and/or the display component 402 depicted in FIG. 4). Further, the one or more control signals can include data used to generate the passenger state query on the display device of the vehicle 108.

The passenger state query can include requesting at least one of the one or more passengers to provide an audible response to the passenger state query, requesting at least one of the one or more passengers to gesture in response to the passenger state query, and/or requesting at least one of the one or more passengers to provide a physical input to an interface of the vehicle (e.g., requesting a passenger of the vehicle 108 to provide a response to a passenger state query by touching a portion of a touchscreen display of the vehicle).

In some embodiments, generating a passenger state query including a request for passenger state feedback based at least in part on the one or more states of the one or more passengers can be used in determining, based at least in part on the sensor data and the vehicle data, when the one or more states of the one or more passengers are associated with one or more health conditions as described in 504 of the method 500 depicted in FIG. 5.

At 804, the method 800 can include determining the one or more states of the one or more passengers based at least in part on the passenger state feedback. For example, the vehicle computing system 112 can determine that one or more states of a passenger of the vehicle 108 are associated with a dyspneic state when the passenger provides an affirmative response to the passenger state query “Do you have an upset stomach?” that is generated on a display device of the vehicle 108.

In some embodiments, determining the one or more states of the one or more passengers based at least in part on the passenger state feedback can be used in determining, based at least in part on the sensor data and the vehicle data, when the one or more states of the one or more passengers are associated with one or more health conditions as described in 504 of the method 500 depicted in FIG. 5.

At 806, the method 800 can include determining that the one or more states of the one or more passengers are associated with at least one of the one or more health conditions when a predetermined time interval has elapsed without receiving the passenger state feedback. For example, the vehicle computing system 112 can determine that the one or more states of a passenger of the vehicle 108 are associated with one or more health conditions when the passenger does not provide passenger state feedback (e.g., a verbal response) within ten seconds.

In some embodiments, determining that the one or more states of the one or more passengers are associated with at least one of the one or more health conditions when a predetermined time interval has elapsed without receiving the passenger state feedback can be used in determining, based at least in part on the sensor data and the vehicle data, when the one or more states of the one or more passengers are associated with one or more health conditions as described in 504 of the method 500 depicted in FIG. 5.

FIG. 9 depicts a flow diagram of an example method of passenger monitoring and intervention according to example embodiments of the present disclosure. One or more portions of a method 900 can be implemented by one or more devices (e.g., one or more computing devices) or systems including, for example, the operations computing system 104, the vehicle 108, or the vehicle computing system 112, shown in FIG. 1. Moreover, one or more portions of the method 900 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 9 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 902, the method 900 can include determining a location of the vehicle. For example, the vehicle computing system 112 can send one or more control signals to the positioning system 118 and/or a navigation device (e.g., a GPS) of the vehicle 108 that can be used to determine the location of the vehicle.

In some embodiments, determining a location of the vehicle can be used in generating one or more control signals to control performance of the one or more actions by the vehicle as described in 508 of the method 500 depicted in FIG. 5.

At 904, the method 900 can include sending one or more notifications to an emergency medical provider. The one or more notifications can include the location of the vehicle and/or the one or more states of the one or more passengers. For example, the vehicle computing system 112 can send one or more control signals to activate the communications system 136 which can send one or more signals to a remote computing system associated with an emergency medical provider including a hospital, a medical clinic, or a personal medical contact (e.g., an individual who can provide medical assistance or contact an emergency medical provider).

In some embodiments, sending one or more notifications to an emergency medical provider can be used in generating one or more control signals to control performance of the one or more actions by the vehicle as described in 508 of the method 500 depicted in FIG. 5.

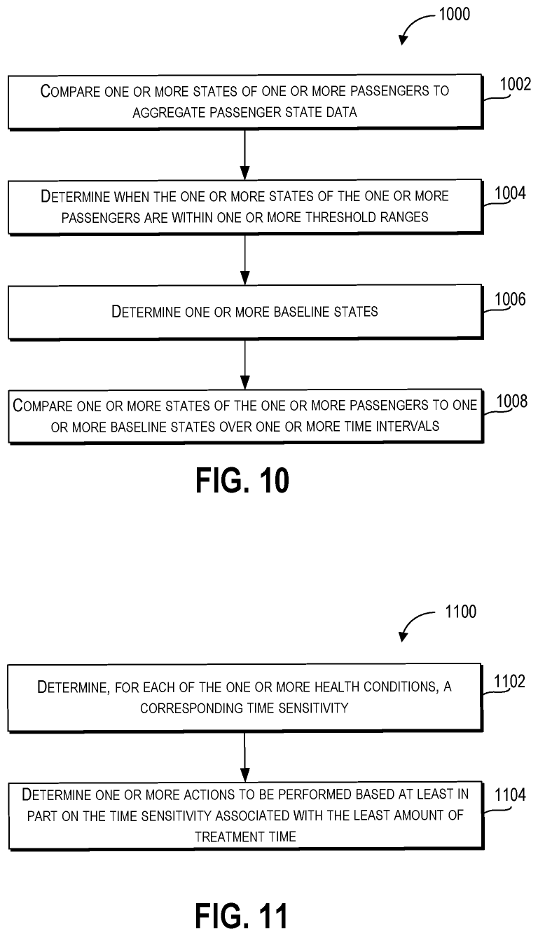

FIG. 10 depicts a flow diagram of an example method of passenger monitoring and intervention according to example embodiments of the present disclosure. One or more portions of a method 1000 can be implemented by one or more devices (e.g., one or more computing devices) or systems including, for example, the operations computing system 104, the vehicle 108, or the vehicle computing system 112, shown in FIG. 1. Moreover, one or more portions of the method 1000 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 10 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 1002, the method 1000 can include comparing the one or more states of the one or more passengers to aggregate passenger state data. In some embodiments, the aggregate passenger state data can include one or more physiological states of one or more test passengers and one or more corresponding physical conditions of the one or more test passengers. Further, the aggregate passenger state data can include one or more states corresponding to any of the one or more states of the one or more passengers included in the sensor data (e.g., heart rates, respiratory rates, blood pressure, movement patterns, and/or body temperature).

Comparisons of the one or more states of the one or more passengers to the aggregate passenger state data can include determining one or more differences between the one or more states of the one or more passengers and corresponding portions of the aggregate passenger state data including. Furthermore, the comparisons of the one or more states of the one or more passengers to the aggregate passenger state data can include determining one or more differences between one or more average values, median values, and/or variance values associated with the one or more states of the one or more passengers and the aggregate passenger state data.

For example the vehicle computing system 112 can compare one or more attributes, parameters, and/or values associated with the one or more states of the one or more passengers to one or more corresponding attributes, parameters, and/or values associated with one or more states (e.g., one or more physiological states) of one or more test passengers (e.g., one or more persons who have agreed to share data associated with their physiological state including their physiological state during a ride in a test vehicle).

In some embodiments, comparing the one or more states of the one or more passengers to aggregate passenger state data can be used in determining, based at least in part on the sensor data and the vehicle data, when the one or more states of the one or more passengers are associated with one or more health conditions as described in 504 of the method 500 depicted in FIG. 5.

At 1004, the method 1000 can include determining when the one or more states of the one or more passengers (e.g., the one or more states of the one or more passengers of the method 500) are within one or more threshold ranges corresponding to the one or more physiological states of the one or more test passengers. For example, the vehicle computing system 112 can determine when a respiratory rate of a passenger is within a threshold respiratory rate. In particular, the vehicle computing system 112 can determine an age range for a passenger of the vehicle 108 (e.g., an age range of a plurality of age ranges including age ranges below the age of five years old, an age range between the ages of five and twelve years old, an age range between the age of twelve and eighty years old, and an age range over the age of eighty) and the threshold respiratory rate range corresponding to the age range (e.g., a threshold respiratory rate range of twelve to twenty-four breaths per minute). The vehicle computing system 112 can then determine when a passenger's respiratory rate is within the threshold respiratory rate range.

In some embodiments, determining when the one or more states of the one or more passengers are within one or more threshold ranges corresponding to the one or more physiological states of the one or more test passengers can be used in determining, based at least in part on the sensor data and the vehicle data, when the one or more states of the one or more passengers are associated with one or more health conditions as described in 504 of the method 500 depicted in FIG. 5.

At 1006, the method 1000 can include determining one or more baseline states corresponding to the one or more states of each of the one or more passengers when the one or more passengers enter the vehicle. For example, the vehicle computing system 112 can determine, based in part on sensor outputs from the one or more sensors 114, one or more baseline states including one or more heart rates, one or more respiratory rates, one or more blood pressures, one or more body temperatures, one or more vocal characteristics (e.g., vocal tone including whether speech is slurred or delayed), one or more movement patterns (e.g., the velocity of a passenger moving their limbs and/or head); and/or any of the one or more states of the one or more passengers that are included in the sensor data.

In some embodiments, determining one or more baseline states corresponding to the one or more states of each of the one or more passengers when the one or more passengers enter the vehicle can be used in determining, based at least in part on the sensor data and the vehicle data, when the one or more states of the one or more passengers are associated with one or more health conditions as described in 504 of the method 500 depicted in FIG. 5.

At 1008, the method 1000 can include comparing the one or more states of the one or more passengers to the one or more baseline states of the one or more passengers at one or more time intervals after the one or more passengers enter the vehicle.

Comparisons of the one or more states of the one or more passengers to the one or more baseline states of the one or more passengers at one or more time intervals after the one or more passengers enter the vehicle can include determining one or more differences and/or similarities between the one or more states of the one or more passengers and corresponding portions of the one or more baseline states of the one or more passengers at one or more time intervals after the one or more passengers enter the vehicle including. Furthermore, the comparisons of the one or more states of the one or more passengers to the one or more baseline states of the one or more passengers at one or more time intervals after the one or more passengers enter the vehicle can include determining one or more differences between one or more average values, median values, and/or variance values associated with the one or more states of the one or more passengers and the one or more baseline states of the one or more passengers at one or more time intervals after the one or more passengers enter the vehicle.

For example the vehicle computing system 112 can compare one or more attributes, parameters, and/or values associated with the one or more states of the one or more passengers to one or more corresponding attributes, parameters, and/or values associated with one or more baseline states (e.g., one or more physiological states) of the one or more passengers at one or more time intervals after the one or more passengers enter the vehicle.

In some embodiments, comparing the one or more states of the one or more passengers to the one or more baseline states of the one or more passengers at one or more time intervals after the one or more passengers enter the vehicle can be used in determining, based at least in part on the sensor data and the vehicle data, when the one or more states of the one or more passengers are associated with one or more health conditions as described in 504 of the method 500 depicted in FIG. 5.

FIG. 11 depicts a flow diagram of an example method of passenger monitoring and intervention according to example embodiments of the present disclosure. One or more portions of a method 1100 can be implemented by one or more devices (e.g., one or more computing devices) or systems including, for example, the operations computing system 104, the vehicle 108, or the vehicle computing system 112, shown in FIG. 1. Moreover, one or more portions of the method 1100 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 11 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 1102, the method 1100 can include determining, for each of the one or more health conditions associated with the one or more states of the one or more passengers, a corresponding time sensitivity that can be associated with an amount of time before requiring medical assistance. In some embodiments, the time sensitivity can include a time to begin treatment of a health condition and/or a time to complete treatment of a health condition.

For example, the vehicle computing system 112 can, after determining the one or more health conditions associated with the one or more states of the one or more passengers access a dataset (e.g., a dataset including a lookup table) including each of the one or more health conditions associated with a corresponding time sensitivity (e.g., a recommended or estimated time in seconds to receive treatment for a health condition).

In some embodiments, determining, for each of the one or more health conditions associated with the one or more states of the one or more passengers, a corresponding time sensitivity associated with an amount of time before requiring medical assistance can be used in determining, based at least in part on the one or more health conditions associated with the one or more passengers, one or more actions to be performed by the vehicle as described in 506 of the method 500 depicted in FIG. 5.

At 1104, the method 1100 can include determining the one or more actions to be performed by the vehicle based at least in part on the time sensitivity associated with the least amount of time before requiring medical assistance. The one or more actions performed by the vehicle can include any of the one or more actions described in 508 of the method 500 depicted in FIG. 5. For example, the vehicle computing system 112 can determine, based on accessing a dataset including the one or more health conditions and corresponding time sensitivities the health condition that has a time sensitivity associated with the least amount of time before requiring treatment. By way of further example, when the vehicle computing system 112 determines two health conditions with respective time sensitivities of five minutes and two hours, the vehicle computing system 112 can determine that in the first five minutes after determining the one or more health conditions, the one or more actions performed by the vehicle 108 will include one or more actions directed to the health condition with the time sensitivity of five minutes.

In some embodiments, determining the one or more actions to be performed by the vehicle based at least in part on the time sensitivity associated with the least amount of time before requiring medical assistance can be used in determining, based at least in part on the one or more health conditions associated with the one or more passengers, one or more actions to be performed by the vehicle as described in 506 of the method 500 depicted in FIG. 5.

FIG. 12 depicts a flow diagram of an example method of passenger monitoring and intervention according to example embodiments of the present disclosure. One or more portions of a method 1200 can be implemented by one or more devices (e.g., one or more computing devices) or systems including, for example, the operations computing system 104, the vehicle 108, or the vehicle computing system 112, shown in FIG. 1. Moreover, one or more portions of the method 1200 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 12 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 1202, the method 1200 can include sending the sensor data (e.g., the sensor data of the method 500) and/or the vehicle data (e.g., the vehicle data of the method 500) to a machine-learned model (e.g., a machine-learned passenger state model). In some embodiments, the machine-learned passenger state model can be trained to receive the sensor data and the vehicle data and provide an output including one or more health condition predictions associated with the one or more health conditions. Further, the machine-learned passenger state model can include one or more features of the one or more machine-learned models 1430 and/or the one or more machine-learned models 1470. For example, the machine-learned model 1470 can receive the sensor data and/or the vehicle data from the one or more sensors 114 of the vehicle 108 and provide an output (e.g., output data) including one or more health condition predictions associated with the one or more health conditions.

In some embodiments, the machine-learned model can be generated and/or trained using training data including a plurality of classified features and a plurality of classified health condition labels. Further, the plurality of classified features can be extracted from one or more sensor outputs (e.g., biometric sensor outputs including heart rate, blood pressure, respiratory rate, and/or body temperature) received from one or more sensors (e.g., the one or more sensors 114 of the vehicle 108) that are used to detect one or more states of a person (e.g., a passenger of a vehicle 108).

When the machine-learned model has been trained, the machine-learned model can associate the plurality of classified features with one or more classified health condition labels that are used to classify or categorize the state of a person associated with the plurality of classified features. In some embodiments, as part of the process of training the machine-learned model, the differences in correct classification output between a machine-learned model (that outputs the one or more classified health condition labels) and a set of classified health condition labels associated with training data that has previously been correctly identified (e.g., ground truth labels), can be processed using an error loss function that can determine a set of probability distributions based on repeated classification of the same set of training data. As such, the effectiveness (e.g., the rate of correct identification of health conditions) of the machine-learned model can be improved over time.

The vehicle computing system can access the machine-learned model in a variety of ways including exchanging (sending and/or receiving via a network) data or information associated with a machine-learned model that is stored on a remote computing device (e.g., the operations computing system 104 and/or the remote computing devices 106); and/or accessing a machine-learned model that is stored locally (e.g., in one or more storage devices of the vehicle computing system 112 and/or the vehicle 108).

The plurality of classified features can be associated with one or more values that can be analyzed individually and/or in various aggregations by the vehicle computing system 112. Analysis of the one or more values associated with the plurality of classified features can include determining a mean, mode, median, variance, standard deviation, maximum, minimum, and/or frequency of the one or more values associated with the plurality of classified features. Further, processing and/or analysis of the one or more values associated with the plurality of classified features can include comparisons of the differences or similarities between the one or more values. For example, one or more body temperatures associated with a health condition experienced by a passenger (e.g., an elevated body temperature associated with a fever) can be associated with a range of body temperatures that are different from the body temperature associated with a passenger that is in a state of good health.

In some embodiments, the plurality of classified features can include a range of sounds associated with a plurality of training subjects (e.g., people who have volunteered to provide test data based on their physical responses), a range of temperatures associated with the plurality of training subjects, a range of body positions associated with the plurality of training subjects, a range of gestures and/or movement patterns associated with the plurality of training subjects, a range of heart rates associated with the plurality of training subjects, a range of respiratory rates associated with the plurality of training subjects, a range of blood pressures associated with the plurality of training subjects, physical characteristics (e.g., age, gender, height, weight, and/or mass) of the plurality of training subjects. The plurality of classified features can be based at least in part on the output from one or more sensors (e.g., the one or more sensors 114) that have captured sensor data from the plurality of training subjects at various times of day and/or in different vehicle conditions (e.g., a warm passenger compartment, a cold passenger compartment, a passenger compartment moving at high velocity, and/or a crowded passenger compartment) and/or environmental conditions (e.g., bright sunlight, rain, overcast conditions, darkness, and/or thunder storms).

Furthermore, the machine-learned model can be generated based at least in part on one or more classification processes or classification techniques. The one or more classification processes or classification techniques can include one or more computing processes performed by one or more computing devices based at least in part on sensor data (e.g., the sensor data of the method 500) associated with physical outputs from a sensor device (e.g., the one or more sensors 114). The one or more computing processes can include the classification (e.g., allocation or sorting into different groups or categories) of the physical outputs from the sensor device, based at least in part on one or more classification criteria associated with one or more health conditions. In some embodiments, the machine-learned model can include a convolutional neural network, a recurrent neural network, a recursive neural network, gradient boosting, a support vector machine, and/or a logistic regression classifier.

In some embodiments, sending the sensor data and the vehicle data to a machine-learned passenger state model can be used in determining, based at least in part on the sensor data and the vehicle data, when the one or more states of the one or more passengers are associated with one or more health conditions as described in 504 of the method 500 depicted in FIG. 5.

At 1204, the method 1200 can include receiving, from the machine-learned passenger state model, the output including the one or more health condition predictions associated with the one or more health conditions (e.g., the one or more health conditions of the method 500). For example, the vehicle computing system 112 can receive one or more machine-learned model classified object labels including one or more health condition predictions from the machine-learned passenger state model. Furthermore, the one or more machine-learned model classified object labels can include one or more probabilities associated with each of the one or more health condition predictions. For example, the vehicle computing system 112 can generate a health condition prediction that a passenger is experiencing a seizure that is associated with a predictive accuracy value of eighty percent (e.g., the health condition prediction is estimated to have an eighty percent probability of being accurate).

In some embodiments, receiving, from the machine-learned passenger state model, the output including the one or more health condition predictions associated with the one or more health conditions can be used in determining, based at least in part on the sensor data and the vehicle data, when the one or more states of the one or more passengers are associated with one or more health conditions as described in 504 of the method 500 depicted in FIG. 5.

FIG. 13 depicts a flow diagram of an example method of passenger monitoring and intervention according to example embodiments of the present disclosure. One or more portions of a method 1300 can be implemented by one or more devices (e.g., one or more computing devices) or systems including, for example, the operations computing system 104, the vehicle 108, or the vehicle computing system 112, shown in FIG. 1. Moreover, one or more portions of the method 1300 can be implemented as an algorithm on the hardware components of the devices described herein (e.g., as in FIG. 1) to, for example, determine one or more health conditions associated with the state of one or more passengers and determine one or more actions to be performed by a vehicle based at least in part on the determined one or more health conditions. FIG. 13 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure.

At 1302, the method 1300 can include determining a respiratory pattern for each of the one or more passengers. Determining the respiratory pattern can be based at least in part on one or more changes in air pressure inside the passenger compartment of the vehicle and/or one or more sensor outputs of a motion sensing respiration sensor in a safety restraint device of the vehicle. In some embodiments, the respiratory pattern can include a rate of respiration (e.g., inhalations, exhalations, or breaths per minute), a respiratory rhythm, a time between inhalation and exhalation, a time between exhalation and inhalation, inspiratory volume, an amount of inspiratory expansion of a passenger's chest, an amount of carbon dioxide exhaled, and/or a velocity of exhaled air. Further, the respiratory pattern can be associated with one or more respiratory behaviors including coughing, wheezing, sneezing, choking, and/or gagging.

For example, the vehicle computing system 112 can receive one or more sensor outputs from the one or more sensors 114 which can include an air-pressure sensor located in the passenger compartment of the vehicle 108 that can detect changes in air pressure in the passenger compartment and can be used by the vehicle computing system 112 to determine a respiratory pattern for each passenger in the passenger compartment of the vehicle 108. Further, the vehicle computing system 112 can determine when changes in the respiratory pattern of a passenger are due to external factors including opening or closing windows or doors of the vehicle 108, adjusting the temperature of the passenger compartment of the vehicle 108, and/or characteristics of the one or more passengers including the age, size, weight, mass, and/or gender of each passenger. By way of further example, the vehicle computing system 112 can receive one or more sensor outputs from the one or more sensors 114 including one or more sensors located in a seating area and/or a seatbelt of the vehicle 108 which can include one or more tactile and/or pressure sensors that can be used to determine the respiratory pattern of a passenger. The vehicle computing system 112 can then determine the respiratory pattern of a passenger based at least in part on the one or more sensor outputs of the one or more sensors located in the seating area and/or the seatbelt of the vehicle 108.

In some embodiments, determining a respiratory pattern for each of the one or more passengers can be used in determining, based at least in part on the sensor data and/or the vehicle data, when the one or more states of the one or more passengers are associated with one or more health conditions (e.g., determining as described in 504 of the method 500 depicted in FIG. 5).

At 1304, the method 1300 can include comparing the respiratory pattern for each of the one or more passengers to a set of respiratory rates associated with the one or more health conditions. Comparisons of the respiratory pattern for each of the one or more passengers to the set of respiratory rates associated with one or more health conditions can include determining one or more differences and/or similarities between the respiratory pattern for each of the one or more passengers and corresponding portions of the set of respiratory rates associated with the one or more health conditions. Furthermore, the comparisons of the respiratory pattern for each of the one or more passengers to the set of respiratory rates associated with one or more health conditions can include determining one or more differences between one or more average values, median values, and/or variance values associated with the respiratory pattern for each of the one or more passengers to the set of respiratory rates associated with one or more health conditions.

For example the vehicle computing system 112 can compare one or more attributes, parameters, and/or values associated with the respiratory pattern for each of the one or more passengers to one or more corresponding attributes, parameters, and/or values associated with a set of respiratory rates associated with the one or more health conditions.

In some embodiments, comparing the respiratory pattern for each of the one or more passengers to a set of respiratory rates associated with the one or more health conditions can be used in determining, based at least in part on the sensor data and the vehicle data, when the one or more states of the one or more passengers are associated with one or more health conditions (e.g., determining as described in 504 of the method 500 depicted in FIG. 5).

FIG. 14 depicts a block diagram of an example computing system 1400 according to example embodiments of the present disclosure. The example computing system 1400 includes a computing system 1410 and a machine learning computing system 1450 that are communicatively coupled over a network 1440. Moreover, the computing system 1400 can include one or more features, functions, devices, elements, and/or components of the system 100 and can perform one or more of the techniques, functions, and/or operations described herein.

In some implementations, the computing system 1410 can perform various operations including receiving sensor data and/or vehicle data from sensors of a vehicle (e.g., the vehicle 108) or one or more remote computing devices, determining states of passengers in the vehicle based on the sensor data and vehicle data, determining health conditions associated with the passengers of the vehicle, and generating control signals to control performance of actions by the vehicle in response to the health conditions. In some implementations, the computing system 1410 can be included in an autonomous vehicle. For example, the computing system 1410 can be on-board the autonomous vehicle. In other implementations, the computing system 1410 is not located on-board the autonomous vehicle. For example, the computing system 1410 can operate offline to perform operations including receiving sensor data and vehicle data from sensors of a vehicle (e.g., the vehicle 108) or one or more remote computing devices, determining states of passengers in the vehicle based on the sensor data and vehicle data, determining health conditions associated with the passengers of the vehicle, and generating control signals to control performance of actions by the vehicle in response to the health conditions. Further, the computing system 1410 can include one or more distinct physical computing devices.

The computing system 1410 includes one or more processors 1412 and a memory 1414. The one or more processors 1412 can be any suitable processing device (e.g., a processor core, a microprocessor, an ASIC, a FPGA, a controller, and/or a microcontroller) and can be one processor or a plurality of processors that are operatively connected. The memory 1414 can include one or more non-transitory computer-readable storage media, including RAM, ROM, EEPROM, EPROM, one or more memory devices, flash memory devices, and/or combinations thereof.

The memory 1414 can store information that can be accessed by the one or more processors 1412. For instance, the memory 1414 (e.g., one or more non-transitory computer-readable storage mediums, memory devices) can store data 1416 that can be obtained, received, accessed, written, manipulated, created, and/or stored. The data 1416 can include, for instance, data associated with operations described herein including receiving sensor data and vehicle data from sensors of a vehicle (e.g., the vehicle 108) or one or more remote computing devices, determining states of passengers in the vehicle based on the sensor data and vehicle data, determining health conditions associated with the passengers of the vehicle, and generating control signals to control performance of actions by the vehicle in response to the health conditions. In some implementations, the computing system 1410 can obtain data from one or more memory devices that are remote from the system 1410.

The memory 1414 can also store computer-readable instructions 1418 that can be executed by the one or more processors 1412. The instructions 1418 can be software written in any suitable programming language or can be implemented in hardware. Additionally, or alternatively, the instructions 1418 can be executed in logically and/or virtually separate threads on the one or more processors 1412.

For example, the memory 1414 can store instructions 1418 that when executed by the one or more processors 1412 cause the one or more processors 1412 to perform any of the operations and/or functions described herein, including, for example, determining one or more states of a vehicle (e.g., the vehicle 108) and/or determining one or more states of one or more passengers of the vehicle including the passenger's biometric or physiological states (e.g., heart rate, blood pressure, and/or respiratory rate).

According to an aspect of the present disclosure, the computing system 1410 can store or include one or more machine-learned models 1430. As examples, the machine-learned models 1430 can be or can otherwise include various machine-learned models including, for example, neural networks (e.g., deep neural networks), support vector machines, decision trees, ensemble models, k-nearest neighbors models, Bayesian networks, or other types of models including linear models and/or non-linear models. Example neural networks include feed-forward neural networks, recurrent neural networks (e.g., long short-term memory recurrent neural networks), convolutional neural networks, or other forms of neural networks.

In some implementations, the computing system 1410 can receive the one or more machine-learned models 1430 from the machine learning computing system 1450 over the network 1440 and can store the one or more machine-learned models 1430 in the memory 1414. The computing system 1410 can then use or otherwise implement the one or more machine-learned models 1430 (e.g., by the one or more processors 1412). In particular, the computing system 1410 can implement the one or more machine-learned models 1430 to determine a state of a vehicle (e.g., the vehicle 108) and/or determine a state of a passenger of the vehicle including the passenger's biometric or physiological state which can be used to determine the occurrence of one or health conditions in the passenger.

The machine learning computing system 1450 includes one or more processors 1452 and a memory 1454. The one or more processors 1452 can be any suitable processing device (e.g., a processor core, a microprocessor, an ASIC, a FPGA, a controller, and/or a microcontroller) and can be one processor or a plurality of processors that are operatively connected. The memory 1454 can include one or more non-transitory computer-readable storage media, including RAM, ROM, EEPROM, EPROM, one or more memory devices, flash memory devices, and/or combinations thereof.

The memory 1454 can store information that can be accessed by the one or more processors 1452. For instance, the memory 1454 (e.g., one or more non-transitory computer-readable storage mediums, memory devices) can store data 1456 that can be obtained, received, accessed, written, manipulated, created, and/or stored. The data 1456 can include, for instance, data associated with operations described herein including receiving sensor data and/or vehicle data from sensors of a vehicle (e.g., the vehicle 108) or one or more remote computing devices, determining states of passengers in the vehicle based on the sensor data and/or vehicle data, determining health conditions associated with the passengers of the vehicle, and generating control signals to control performance of actions by the vehicle in response to the health conditions. In some implementations, the machine learning computing system 1450 can obtain data from one or more memory devices that are remote from the system 1450.

The memory 1454 can also store computer-readable instructions 1458 that can be executed by the one or more processors 1452. The instructions 1458 can be software written in any suitable programming language or can be implemented in hardware. Additionally, or alternatively, the instructions 1458 can be executed in logically and/or virtually separate threads on the one or more processors 1452.

For example, the memory 1454 can store instructions 1458 that when executed by the one or more processors 1452 cause the one or more processors 1452 to perform any of the operations and/or functions described herein, including, for example, receiving sensor data and vehicle data from sensors of a vehicle (e.g., the vehicle 108) or one or more remote computing devices, determining states of passengers in the vehicle based on the sensor data and vehicle data, determining health conditions associated with the passengers of the vehicle, and generating control signals to control performance of actions by the vehicle in response to the health conditions.

In some implementations, the machine learning computing system 1450 includes one or more server computing devices. If the machine learning computing system 1450 includes multiple server computing devices, such server computing devices can operate according to various computing architectures, including, for example, sequential computing architectures, parallel computing architectures, or some combination thereof.

In addition or alternatively to the one or more machine-learned models 1430 at the computing system 1410, the machine learning computing system 1450 can include one or more machine-learned models 1470. As examples, the one or more machine-learned models 1470 can be or can otherwise include various machine-learned models including, for example, neural networks (e.g., deep neural networks), support vector machines, decision trees, ensemble models, k-nearest neighbors models, Bayesian networks, or other types of models including linear models and/or non-linear models. Example neural networks include feed-forward neural networks, recurrent neural networks (e.g., long short-term memory recurrent neural networks), convolutional neural networks, or other forms of neural networks.

As an example, the machine learning computing system 1450 can communicate with the computing system 1410 according to a client-server relationship. For example, the machine learning computing system 1450 can implement the one or more machine-learned models 1470 to provide a web service to the computing system 1410. For example, the web service can perform one or more operations including determining a state of a vehicle (e.g., the vehicle 108) and/or determining a state of a passenger's biometric or physiological state which can be used to determine the occurrence of one or health conditions in the passenger.

As such, the one or more machine-learned models 1430 can located and used at the computing system 1410 and/or the one or more machine-learned models 1470 can be located and used at the machine learning computing system 1450.

In some implementations, the machine learning computing system 1450 and/or the computing system 1410 can train the machine-learned models 1430 and/or 1470 through use of a model trainer 1480. The model trainer 1480 can train the machine-learned models 1430 and/or 1470 using one or more training or learning algorithms. One example training technique is backwards propagation of errors. In some implementations, the model trainer 1480 can perform supervised training techniques using a set of labeled training data. In other implementations, the model trainer 1480 can perform unsupervised training techniques using a set of unlabeled training data. The model trainer 1480 can perform a number of generalization techniques to improve the generalization capability of the models being trained. Generalization techniques include weight decays, dropouts, or other techniques.

In particular, the model trainer 1480 can train the one or more machine-learned models 1430 and/or the one or more machine-learned models 1470 based on a set of training data 1482. The training data 1482 can include, for example, data associated with various characteristics and/or properties of test passengers (e.g., passengers of different ages, sizes, weights, masses, somatotypes, and/or health conditions) collected from persons who volunteered the use of data associated with their physical states (e.g., heart rates, mass, weight, height, and/or subjective feedback). The model trainer 1480 can be implemented in hardware, firmware, and/or software controlling one or more processors.

The computing system 1410 can also include a network interface 1420 used to communicate with one or more systems or devices, including systems or devices that are remotely located from the computing system 1410. The network interface 1420 can include any circuits, components, and/or software, for communicating with one or more networks (e.g., the network 1440). In some implementations, the network interface 1420 can include, for example, one or more of a communications controller, receiver, transceiver, transmitter, port, conductors, software and/or hardware for communicating data. Similarly, the machine learning computing system 1450 can include a network interface 1460.

The networks 1440 can be any type of network or combination of networks that allows for communication between devices. In some embodiments, the network 1440 can include one or more of a local area network, wide area network, the Internet, secure network, cellular network, mesh network, peer-to-peer communication link and/or some combination thereof and can include any number of wired or wireless links. Communication over the network 1440 can be accomplished, for instance, via a network interface using any type of protocol, protection scheme, encoding, format, and/or packaging.

FIG. 14 illustrates one example computing system 1400 that can be used to implement the present disclosure. Other computing systems can be used as well. For example, in some implementations, the computing system 1410 can include the model trainer 1480 and the training dataset 1482. In such implementations, the machine-learned models 1430 can be both trained and used locally at the computing system 1410. As another example, in some implementations, the computing system 1410 is not connected to other computing systems.

In addition, components illustrated and/or discussed as being included in one of the computing systems 1410 or 1450 can instead be included in another of the computing systems 1410 or 1450. Such configurations can be implemented without deviating from the scope of the present disclosure. The use of computer-based systems allows for a great variety of possible configurations, combinations, and divisions of tasks and functionality between and among components. Computer-implemented operations can be performed on a single component or across multiple components. Computer-implemented tasks and/or operations can be performed sequentially or in parallel. Data and instructions can be stored in a single memory device or across multiple memory devices.

While the present subject matter has been described in detail with respect to specific example embodiments and methods thereof, it will be appreciated that those skilled in the art, upon attaining an understanding of the foregoing can readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A computer-implemented method of autonomous vehicle operation, the computer-implemented method comprising:
receiving, by a computing system comprising one or more computing devices, sensor data and vehicle data, from one or more sensors of a vehicle, wherein the sensor data is associated with one or more states of one or more passengers in a passenger compartment of the vehicle, and wherein the vehicle data is associated with one or more states of the vehicle;
determining, by the computing system, based at least in part on the sensor data and the vehicle data, when the one or more states of the one or more passengers are associated with one or more health conditions;
determining, by the computing system, based at least in part on the one or more health conditions associated with the one or more passengers, one or more actions to be performed by the vehicle; and
generating, by the computing system, one or more control signals to control performance of the one or more actions by the vehicle.

2. The computer-implemented method of claim 1, wherein determining, by the computing system, based at least in part on the sensor data and the vehicle data, when the one or more states of the one or more passengers are associated with one or more health conditions comprises:
determining, by the computing system, when the sensor data satisfies one or more health response criteria associated with an occurrence of the one or more health conditions.

3. The computer-implemented method of claim 2, wherein determining, by the computing system, based at least in part on the sensor data and the vehicle data, when the one or more states of the one or more passengers are associated with one or more health conditions comprises:
adjusting, by the computing system, the one or more health response criteria based at least in part on the vehicle data.

4. The computer-implemented method of claim 2, wherein determining, by the computing system, based at least in part on the sensor data and the vehicle data, when the one or more states of the one or more passengers are associated with one or more health conditions comprises:
determining, by the computing system, a number of the one or more passengers in the vehicle; and
adjusting, by the computing system, the one or more health response criteria based at least in part on the number of the one or more passengers in the vehicle.

5. The computer-implemented method of claim 1, wherein the vehicle data comprises information associated with a velocity of the vehicle, an acceleration of the vehicle, a deceleration of the autonomous vehicle, a centrifugal force on the passenger compartment of the vehicle, a temperature of the passenger compartment of the vehicle, a pressure in the passenger compartment of the vehicle, or a humidity of the passenger compartment of the vehicle.

6. The computer-implemented method of claim 1, wherein the one or more sensors comprise one or more heart rate sensors, one or more respiratory rate sensors, one or more image sensors, one or more microphones, or one or more thermal sensors.

7. The computer-implemented method of claim 1, wherein determining, by the computing system, based at least in part on the sensor data and the vehicle data, when the one or more states of the one or more passengers are associated with one or more health conditions comprises:
determining, by the computing system, based at least in part on the sensor data, one or more characteristics of one or more motions of a passenger of the one or more passengers with respect to a seating area of the passenger in the passenger compartment of the vehicle; and
determining, by the computing system, when the one or more motions of the passenger satisfy one or more passenger motion criteria associated with the one or more health conditions.

8. The computer-implemented method of claim 1, wherein the one or more actions performed by the vehicle comprise sending one or more signals to a medical facility, sending one or more signals to a vehicle tele-operator, sending one or more notifications to an emergency medical contact associated with at least one of the one or more passengers, or driving the vehicle to a destination determined by at least one of the one or more passengers.

9. The computer-implemented method of claim 1, wherein determining, by the computing system, based at least in part on the sensor data and the vehicle data, when the one or more states of the one or more passengers are associated with one or more health conditions comprises:
generating, by the computing system, a passenger state query comprising a request for passenger state feedback based at least in part on the one or more states of the one or more passengers, wherein the passenger state query comprises one or more visual indications or one or more auditory indications; and
determining, by the computing system, the one or more states of the one or more passengers based at least in part on the passenger state feedback.

10. The computer-implemented method of claim 9, wherein the passenger state query comprises requesting at least one of the one or more passengers to provide an audible response to the passenger state query, requesting at least one of the one or more passengers to gesture in response to the passenger state query, or requesting at least one of the one or more passengers to provide a physical input to an interface of the vehicle.

11. The computer-implemented method of claim 9, wherein determining, by the computing system, based at least in part on the sensor data and the vehicle data, when the one or more states of the one or more passengers are associated with one or more health conditions comprises:
determining, by the computing system, that the one or more states of the one or more passengers are associated with at least one of the one or more health conditions when a predetermined time interval has elapsed without receiving the passenger state feedback.

12. The computer-implemented method of claim 1, wherein determining, by the computing system, based at least in part on the sensor data and the vehicle data, when the one or more states of the one or more passengers are associated with one or more health conditions comprises:
comparing, by the computing system, the one or more states of the one or more passengers to aggregate passenger state data comprising one or more physiological states of one or more test passengers and one or more corresponding physical conditions of the one or more test passengers; and
determining, by the computing system, when the one or more states of the one or more passengers are within one or more threshold ranges corresponding to the one or more physiological states of the one or more test passengers.

13. The computer-implemented method of claim 1, wherein determining, by the computing system, based at least in part on the sensor data and the vehicle data, when the one or more states of the one or more passengers are associated with one or more health conditions comprises:
determining, by the computing system, one or more baseline states corresponding to the one or more states of each of the one or more passengers when the one or more passengers enter the vehicle; and
comparing, by the computing system, the one or more states of the one or more passengers to the one or more baseline states of the one or more passengers at one or more time intervals after the one or more passengers enter the vehicle.

14. The computer-implemented method of claim 1, wherein determining, by the computing system, based at least in part on the one or more health conditions associated with the one or more passengers, one or more actions to be performed by the vehicle comprises:
determining, by the computing system, for each of the one or more health conditions associated with the one or more states of the one or more passengers, a corresponding time sensitivity associated with an amount of time before requiring medical assistance; and
determining, by the computing system, the one or more actions to be performed by the vehicle based at least in part on the time sensitivity associated with the least amount of time before requiring medical assistance.

15. One or more tangible non-transitory computer-readable media storing computer-readable instructions that when executed by one or more processors cause the one or more processors to perform operations, the operations comprising:
receiving sensor data and vehicle data, from one or more sensors of a vehicle, wherein the sensor data is associated with one or more states of one or more passengers in a passenger compartment of the vehicle, and wherein the vehicle data is associated with one or more states of the vehicle;
determining, based at least in part on the sensor data and the vehicle data, when the one or more states of the one or more passengers are associated with one or more health conditions;
determining, based at least in part on the one or more health conditions associated with the one or more passengers, one or more actions to be performed by the vehicle; and
generating one or more control signals to control performance of the one or more actions by the vehicle.

16. The one or more tangible non-transitory computer-readable media of claim 15, determining, based at least in part on the sensor data and the vehicle data, when the one or more states of the one or more passengers are associated with one or more health conditions comprises:
sending the sensor data and the vehicle data to a machine-learned passenger state model trained to receive the sensor data and the vehicle data, and provide an output comprising one or more health condition predictions associated with the one or more health conditions; and
receiving, from the machine-learned passenger state model, the output comprising the one or more health condition predictions associated with the one or more health conditions.

17. The one or more tangible non-transitory computer-readable media of claim 15, wherein determining, based at least in part on the sensor data and the vehicle data, when the one or more states of the one or more passengers are associated with one or more health conditions comprises:
determining a respiratory pattern for each of the one or more passengers based at least in part on one or more changes in air pressure inside the passenger compartment of the vehicle or one or more sensor outputs of a motion sensing respiration sensor in a safety restraint device of the vehicle; and
comparing the respiratory pattern for each of the one or more passengers to a set of respiratory rates associated with the one or more health conditions.

18. A vehicle comprising:
one or more processors;
a memory comprising one or more computer-readable media, the memory storing computer-readable instructions that when executed by the one or more processors cause the one or more processors to perform operations comprising:
receiving sensor data and vehicle data, from one or more sensors of a vehicle, wherein the sensor data is associated with one or more states of one or more passengers in a passenger compartment of the vehicle, and wherein the vehicle data is associated with one or more states of the vehicle;
determining, based at least in part on the sensor data and the vehicle data, when the one or more states of the one or more passengers are associated with one or more health conditions;
determining, based at least in part on the one or more health conditions associated with the one or more passengers, one or more actions to be performed by the vehicle; and
generating one or more control signals to control performance of the one or more actions by the vehicle.

19. The vehicle of claim 18, wherein generating one or more control signals to control performance of the one or more actions by the vehicle comprises:
determining a location of the vehicle; and
sending one or more notifications to an emergency medical provider, wherein the one or more notifications comprise the location of the vehicle and the one or more states of the one or more passengers.

20. The vehicle of claim 18, wherein the one or more health conditions comprise a dyspneic state, a state of emesis, a seizure state, a cardiac arrest state, or a stroke state.
